SEQUENCE LISTING (35 sequences)

SEQ ID NO: 1 - DNA, 20 bp, Artificial Sequence
  Synthetic Cpf1-39-56-sp1
gttgcattaatcagcatcag

SEQ ID NO: 2 - DNA, 20 bp, Artificial Sequence
  Synthetic Cpf1-39-56-sp2
cgcccttgaagttccttctg

SEQ ID NO: 3 - DNA, 20 bp, Artificial Sequence
  Synthetic Cpf1-FarP7K-sp1
tccactccaagatgctccat

SEQ ID NO: 4 - DNA, 20 bp, Artificial Sequence
  Synthetic Cpf1-FarP7K-sp2
aaaccgttcaagagtttttg

SEQ ID NO: 5 - DNA, 20 bp, Artificial Sequence
  Synthetic Cpf1-FarP7K-sp3
aatttagcactcgtggagat

SEQ ID NO: 6 - DNA, 20 bp, Artificial Sequence
  Synthetic Cpf1-FarP7K-sp4
tcgcccgaatgaatccagtt

SEQ ID NO: 7 - DNA, 20 bp, Artificial Sequence
  Synthetic Cpf1-FarP7K-sp5
ggaagaatccgttaatcgtc

SEQ ID NO: 8 - DNA, 20 bp, Artificial Sequence
  Synthetic Cpf1-FarP7K-sp6
ccagtgagttttcacacgaa

SEQ ID NO: 9 - DNA, 20 bp, Artificial Sequence
  Synthetic Cpf1-FarP18K-sp1
cactgatgaagaaacggtgt

SEQ ID NO: 10 - DNA, 20 bp, Artificial Sequence
  Synthetic Cpf1-FarP18K-sp2
tctactgtaatcatgtccca

SEQ ID NO: 11 - DNA, 20 bp, Artificial Sequence
  Synthetic Cpf1-FarP18K-sp3
tcgttggttcattatacacc

SEQ ID NO: 12 - DNA, 20 bp, Artificial Sequence
  Synthetic Cpf1-FarP18K-sp4
gaattaatcgtgctgataca

SEQ ID NO: 13 - DNA, 20 bp, Artificial Sequence
  Synthetic Cpf1-SegF-sp1
ttccttctccaccctgacca

SEQ ID NO: 14 - DNA, 20 bp, Artificial Sequence
  Synthetic Cpf1-SegF-sp2
atgcagatattagctcacgt

SEQ ID NO: 15 - DNA, 20 bp, Artificial Sequence
  Synthetic Cpf1-SegF-sp3
accatcgtattttataatta

SEQ ID NO: 16 - DNA, 20 bp, Artificial Sequence
  Synthetic Cpf1-Hoc-sp1
cagttgatataactcctaaa

SEQ ID NO: 17 - DNA, 20 bp, Artificial Sequence
  Synthetic Cpf1-Hoc-sp2
atcaataacccctgtaggtg

SEQ ID NO: 18 - DNA, 20 bp, Artificial Sequence
  Synthetic Cpf1-Hoc-sp3
gttatgtactaaaaggacct

SEQ ID NO: 19 - DNA, 20 bp, Artificial Sequence
  Synthetic Cpf1-Hoc-sp4
gaaactggtatcatctatac

SEQ ID NO: 20 - DNA, 20 bp, Artificial Sequence
  Synthetic Cpf1-Soc-sp1
agcagaaattagatggaaat

SEQ ID NO: 21 - DNA, 20 bp, Artificial Sequence
  Synthetic Cpf1-Soc-sp2
atattaacataaccgcgagt

SEQ ID NO: 22 - DNA, 20 bp, Artificial Sequence
  Synthetic Cpf1-Soc-sp3
cagcaatccattcagtacgt

SEQ ID NO: 23 - DNA, 20 bp, Artificial Sequence
  Synthetic Cpf1-Soc-sp4
tggaaagtaactggttaata

SEQ ID NO: 24 - DNA, 20 bp, Artificial Sequence
  Synthetic Cpf1-Mrh2-sp1
ttcattacatgtcgtgaaat

SEQ ID NO: 25 - DNA, 20 bp, Artificial Sequence
  Synthetic Cpf1-Mrh2-sp2
gatattatcatttcacgaca

SEQ ID NO: 26 - DNA, 20 bp, Artificial Sequence
  Synthetic Cpf1-Mrh2-sp3
aattcgacttgcttctcacc

SEQ ID NO: 27 - DNA, 20 bp, Artificial Sequence
  Synthetic Cpf1-IPIII-sp1
aagtcggaagcctttgtagc

SEQ ID NO: 28 - DNA, 20 bp, Artificial Sequence
  Synthetic Cpf1-IPIII-sp2
tgcttggcaaattcaagacc

SEQ ID NO: 29 - DNA, 20 bp, Artificial Sequence
  Synthetic Cpf1-IPIII-sp3
ctgatcggtaggtccactca

SEQ ID NO: 30 - DNA, 20 bp, Artificial Sequence
  Synthetic Cpf1-IPIII-sp4
ctacagaagcttcggcaata

SEQ ID NO: 31 - DNA, 20 bp, Artificial Sequence
  Synthetic Cpf1-IPII-sp1
cttctaagttcggcatgtct

SEQ ID NO: 32 - DNA, 20 bp, Artificial Sequence
  Synthetic Cpf1-IPII-sp2
ttacggtctttatcgggcaa

SEQ ID NO: 33 - DNA, 20 bp, Artificial Sequence
  Synthetic Cas9-IPIII-sp1
atggaaaggtcttgatgcaa

SEQ ID NO: 34 - DNA, 20 bp, Artificial Sequence
  Synthetic Cas9-IPIII-sp2
attatcaatgacccatttac

SEQ ID NO: 35 - DNA, 20 bp, Artificial Sequence
  Synthetic Cas9-IPIII-sp3
ggcctttactacagaagctt

(19) United States
(12) Patent Application Publication
(10) Pub. No.: US 2022/0184205 A1
(43) Pub. Date: Jun. 16, 2022

(54) UNIVERSAL BACTERIOPHAGE T4 NANOPARTICLE PLATFORM TO DESIGN MULTIPLEX SARS-COV-2 VACCINE CANDIDATES BY CRISPR ENGINEERING

(71) Applicant: The Catholic University of America, Washington, DC (US)

(72) Inventors: Venigalla B. RAO, Washington, DC (US); Jingen ZHU, Washington, DC (US)

(21) Appl. No.: 17/548,629

(22) Filed: Dec. 13, 2021

Related U.S. Application Data
(60) Provisional application No. 63/126,047, filed on Dec. 16, 2020.

(51) Int. Cl. A61K 39/215; C12N 7/00; C12N 15/86; C12N 15/90

(52) U.S. Cl. CPC A61K 39/215; C12N 7/00; C12N 15/86; C12N 2795/10143; C12N 2310/20; C12N 2770/20034; C12N 2770/20052; C12N 15/902

(57) ABSTRACT

The present disclosure relates to a system for and a method of incorporating SARS-CoV-2 genes and proteins into T4 phages. The present disclosure also relates to vaccine against SARS-CoV-2 containing recombinant T4 phages created using the method provided in the present disclosure.

### CROSS-REFERENCE TO RELATED APPLICATIONS

This application claims benefit of priority of U.S. Provisional Patent Application No. 63/126,047 entitled, “UNIVERSAL BACTERIOPHAGE T4 NANOPARTICLE PLATFORM TO DESIGN MULTIPLEX SARS-COV-2 VACCINE CANDIDATES BY CRISPR ENGINEERING” filed Dec. 16, 2020. The entire contents and disclosures of these patent applications are incorporated herein by reference in their entirety.

### REFERENCE TO A “SEQUENCE LISTING”

The present application includes a Sequence Listing which has been submitted electronically in an ASCII text format. This Sequence Listing is named 109007-23918US01_sequence listing.TXT was created on Nov. 8, 2021, is 7,251 bytes in size and is hereby incorporated by reference in its entirety.

### BACKGROUND

### Field of the Invention

The present disclosure relates to a system for and a method of incorporating SARS-CoV-2 genes and proteins into T4 phages. The present disclosure also relates to vaccine against SARS-CoV-2 containing recombinant T4 phages created using the method provided in the present disclosure.

### BACKGROUND OF THE INVENTION

Rapid discovery of safe and effective vaccines against emerging and pandemic pathogens such as the novel coronavirus SARS-CoV-2 requires a “universal” vaccine design platform that can be adapted to any infectious agent. It should be a multicomponent platform, allowing the incorporation of diverse targets, such as DNAs and proteins. Moreover, the platform would idealy also suitable for the development of multivalent vaccines, incorporating full-length proteins as well as peptides and domains in various combinations. Such a multiplex platform would not only compress the timeline for vaccine discovery but also offers critical choices for selecting the most effective vaccine candidate without going through iterative design cycles.

Though numerous vaccine platforms have been developed, most are limited to single vaccine target, require strong chemical adjuvants to boost immune responses, and lack sufficient engineering flexibility to generate multiplex vaccines. Here, a “universal” multiplex vaccine design is needed.

Tailed bacteriophages such as T4 are the most abundant and widely distributed organisms on Earth. T4 belongs to myoviridae family, infects Escherichia coli, and has served as an extraordinary model in molecular biology and biotechnology. As shown in FIG. 1, it consists of a 1200 Å long and 860 Å wide prolate head (or capsid) (126) that encapsidates a ˜170 kb linear DNA genome (124) and a 1400 Å long contractile tail with six long tail fibers emanating from a baseplate present at the tip of the tail (124). The head, the principal component for vaccine design, is assembled with 155 hexameric capsomers of the major capsid protein gp23* (120), 11 pentamers of gp24* at eleven of twelve vertices, and 1 dodecameric portal protein gp20 at the unique twelfth vertex. The “*” represents cleaved mature form of the capsid proteins.

In addition to these essential components, the T4 capsid is coated with two nonessential proteins; Soc (small outer capsid protein) (118), which is a 9.1 kDa protein and Hoc (122), which is a highly antigenic outer capsid protein with the size of 40.4 kDa, In each capsid, there are 870 copies of Soc and 155 copies of Hoc. Soc is a trimer bound to quasi three-fold axes and acts as a “molecular clamp” by clasping adjacent capsomers. Hoc is a 170 Å-long fiber containing a string of four Ig-like domains with its N-terminal domain exposed at the tip of the fiber. Soc reinforces an already stable T4 capsid while Hoc helps phage to adhere to host surfaces. The structure of T4 is illustrated in FIG. 1.

The above provides an ideal architecture to develop a universal vaccine design template. Therefore, exploration of CRISPR engineering of bacteriophage (phage) T4 into a potentially vaccine development platform that can be applied to any emerging pathogen is highly desirable.

### SUMMARY

According to a first broad aspect of the present disclosure, a universal vaccine design platform comprising: at least one bacterial phage; and at least one host cell comprising at least one CRISPR plasmid and at least one donor plasmid, wherein the bacterial phage can infect the host cell, wherein the CRISPR plasmid comprises a gene encoding at least one endonuclease that can be expressed within the host cell and create a cut in the genome of the bacterial phage, wherein the donor plasmid comprises at least one DNA segment that can be inserted into the genome of the bacterial phage at the cut created by the endonuclease encoded in the CRISPR plasmid, and wherein the genome of the bacterial phage comprising at least one inserted DNA segment from the donor plasmid can be packaged and released from the host cell is provided.

According to a second broad aspect of the present disclosure, a method of producing vaccine comprising: introducing at least one CRISPR plasmid and at least one donor plasmid into at least one host cell; infecting the host cell with at least one bacterial phage; and purifying the recombinant bacterial phage released from the host cell, wherein the CRISPR plasmid comprises a gene encoding at least one endonuclease that can be expressed within the host cell and create a cut in the genome of the bacterial phage, and wherein the donor plasmid comprises at least one DNA segment that can be inserted into the genome of the bacterial phage at the cut created by the endonuclease encoded in the CRISPR plasmid is provided.

According to a third broad aspect of the present disclosure, a vaccine produced using the method and platform above is provided.

According to a fourth aspect of the present disclosure, a vaccine comprising at least one recombinant bacterial phage, wherein the recombinant bacterial phage comprises at least one modification selected from the group consisting of: at least one gene encoding a component of SARS-CoV-2 inserted in the genome of T4 phage, at least one component of SARS-CoV-2 displayed on the surface of T4 phage, and at least one component of SARS-CoV-2 packaged in T4 phage but not inserted in the genome of T4 phage is provided.

Other aspects and features of the present disclosure will become apparent to those skilled in the art upon review of the following description of specific embodiments of the invention in conjunction with the accompanying figures.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 is a graph showing the schematic diagram of T4-SARS-CoV-2 nanovaccine according to an exemplary embodiment of the present disclosure.

FIG. 2 is graph showing the schematic of T4 CRISPR engineering according to an exemplary embodiment of the present disclosure.

FIG. 3 is a graph showing the four nonessential regions of the T4 genome chosen for deletion and insertion of various SARS-CoV-2 genes according to an exemplary embodiment of the present disclosure.

FIG. 4 is a schematic graph showing the 18-kb nonessential segment FarP and 11-kb nonessential segment 39-56 on T4 genome according to an exemplary embodiment of the present disclosure.

FIG. 5 is a photo showing the plaque size of wild-type (WT), T4-FarP 18 kb del., T4-39-56 11 kb del., T4-FarP&39-56 29 kb del. phages according to an exemplary embodiment of the present disclosure.

FIG. 6 is an illustration showing the schematic of SARS-CoV-2 virus, spike trimer, and receptor binding domain (RBD) according to an exemplary embodiment of the present disclosure.

FIG. 7 is a graph showing the S-full length (S-fl) and S-ectodomain (5-ecto) expression cassettes used for insertion into T4 genome according to an exemplary embodiment of the present disclosure.

FIG. 8 is a graph showing the efficiency of plating (EOP) of representative Cpf1-FarP7K and Cpf1-SegF spacers according to an exemplary embodiment of the present disclosure.

FIG. 9 is a photo showing the phage plaque plate from phage infecting bacteria containing Cpf1-FarP7K spacer only, 5-ecto donor only, or Cpf1-FarP7K spacer combined with S-ecto donor according to an exemplary embodiment of the present disclosure.

FIG. 10 is a graph showing the EOP of three sets of Cpf1-FarP7K spacers and three sets of Cpf1-SegF spacers according to an exemplary embodiment of the present disclosure.

FIG. 11 is a graph showing the recombination frequency of three spike genes (RBD, S-ecto, and S-fl) insertion according to an exemplary embodiment of the present disclosure.

FIG. 12 is a graph showing the percentage of the plaques generated in S-ecto recombination contained the correct S-ecto insert according to an exemplary embodiment of the present disclosure.

FIG. 13 is a photo showing the plaque size of wild-type (WT), T4-RBD, T4-S-fl, T4-S-ecto, and T4-(S-ecto)-RBD recombinant phages according to an exemplary embodiment of the present disclosure.

FIG. 14 is a graph showing the EOP of various spacers used for T4 genome engineering according to an exemplary embodiment of the present disclosure.

FIG. 15 is a graph showing the T4 phage head morphogenesis according to an exemplary embodiment of the present disclosure.

FIG. 16 is a graph showing the construction of T4-IPIIIΔ-IPIIΔ-CTSam-NP phage according to an exemplary embodiment of the present disclosure.

FIG. 17 is a photo showing the SDS-PAGE and Western Blotting analysis of phage particles with IPII and IPIII deletions (IPIIΔIPIIIΔ) and NP encapsidation according to an exemplary embodiment of the present disclosure.

FIG. 18 is a photo showing the SDS-PAGE confirming NP expression and encapsidation in both B40 (Sup1) and BL21-RIPL (Sup1) infected with T4-CTSa-NP phage according to an exemplary embodiment of the present disclosure.

FIG. 19 is a photo showing the Western Blot confirming NP expression and encapsidation in both B40 (Sup1) and BL21-RIPL (Sup1) infected with T4-CTSa-NP phage according to an exemplary embodiment of the present disclosure.

FIG. 20 is a photo showing the quantification of the copy number of T4 encapsidated NP protein molecules using WB and NP standard according to an exemplary embodiment of the present disclosure.

FIG. 21 is a graph showing the construction of T4-SocΔ-HocA and T4-SocΔ-(E epitope-Hoc) phages according to an exemplary embodiment of the present disclosure.

FIG. 22 is a photo showing the SDS-PAGE of Hoc deletion and Soc deletion phage (HocASocA) according to an exemplary embodiment of the present disclosure.

FIG. 23 is a figure showing the structural model of the viroporin-like tetrameric assembly of the E protein according to an exemplary embodiment of the present disclosure.

FIG. 24 is a photo showing SDS-PAGE of recombinant phages displaying Ee-Hoc or Ec-Hoc fusion proteins according to an exemplary embodiment of the present disclosure.

FIG. 25 is a graph showing the insertion of Soc-RBD gene into phage genome at the Soc deletion site according to an exemplary embodiment of the present disclosure.

FIG. 26 is a schematic graph showing the inefficient in vivo display of E. coli-expressed Soc-RBD on T4 phage according to an exemplary embodiment of the present disclosure.

FIG. 27 is an SDS-PAGE photo showing the inefficient in vivo display of E. coli-expressed Soc-RBD on T4 phage according to an exemplary embodiment of the present disclosure.

FIG. 28 is a photo showing the solubility analysis of Soc-RBD according to an exemplary embodiment of the present disclosure.

FIG. 29 is a photo showing the analysis of Soc-RBD in supernatant according to an exemplary embodiment of the present disclosure.

FIG. 30 is a graph showing the structural models of WT RBD and various truncated RBDs bound to human ACE2 according to an exemplary embodiment of the present disclosure.

FIG. 31 is a graph showing the solubility analysis of Soc-fused truncated RBDs after cloning and expression in E. coli under the control of the phage T7 promoter according to an exemplary embodiment of the present disclosure.

FIG. 32 is a schematic graph showing the Soc-sRBD or Soc-SpyCatcher (SpyC) in vivo display on T4-SocΔ capsid according to an exemplary embodiment of the present disclosure.

FIG. 33 is an SDS-PAGE photo showing about 100 copies of Soc-sRBD displayed on T4 capsid according to an exemplary embodiment of the present disclosure.

FIG. 34 is a photo showing the solubility analysis of Soc-SpyCatcher according to an exemplary embodiment of the present disclosure.

FIG. 35 is an SDS-PAGE photo showing about 500 copies of Soc-SpyCatcher displayed on T4 capsid according to an exemplary embodiment of the present disclosure.

FIG. 36 is a photo showing the solubility analysis of SUMO-RBD-Spytag according to an exemplary embodiment of the present disclosure.

FIG. 37 is a schematic graph showing the solubilization and refolding of SUMO-RBD-Spytag inclusion body according to an exemplary embodiment of the present disclosure.

FIG. 38 is a photo showing the display of rRBD on the T4-SpyCacher surface at increasing ratios of rRBD molecules to capsid Soc binding sites according to an exemplary embodiment of the present disclosure.

FIG. 39 is a graph showing the comparison of binding of RBD phages to soluble human ACE2 receptor according to an exemplary embodiment of the present disclosure.

FIG. 40 is a graph showing the comparison of binding of RBD phages to soluble human ACE2 receptor at different concentrations of ACE2 according to an exemplary embodiment of the present disclosure.

FIG. 41 is a graph showing the comparison of binding of RBD phages to soluble human mAbl antibody according to an exemplary embodiment of the present disclosure.

FIG. 42 is a graph showing the comparison of binding of RBD phages to soluble human mAb2 antibody according to an exemplary embodiment of the present disclosure.

FIG. 43 is a graph showing the comparison of binding of RBD phages to soluble human pAb antibody according to an exemplary embodiment of the present disclosure.

FIG. 44 is a graph showing the comparison of binding of RBD phages to mAb1 antibody at different concentrations of mAbl antibody according to an exemplary embodiment of the present disclosure.

FIG. 45 is a graph showing the comparison of binding of RBD phages to mAb2 antibody at different concentrations of mAb2 antibody according to an exemplary embodiment of the present disclosure.

FIG. 46 is a graph showing the comparison of binding of RBD phages to pAb antibody at different concentrations of pAb antibody according to an exemplary embodiment of the present disclosure.

FIG. 47 is a graph showing the comparison of binding of E. coli-produced rRBD to human ACE2 with the HEK293- produced RBD according to an exemplary embodiment of the present disclosure.

FIG. 48 is a graph showing the comparison of E. coli-produced rRBD and human HEK293 cell-produced RBD using a panel of RBD-specific mAbs and pAbs. according to an exemplary embodiment of the present disclosure.

FIG. 49 is a graph showing the size-exclusion chromatography (SEC) elution profile of S-ecto-spy trimers according to an exemplary embodiment of the present disclosure.

FIG. 50 is a photo showing the reducing SDS-PAGE (top) and BLUE NATIVE-PAGE (bottom) profiles of SEC-purified trimer fractions according to an exemplary embodiment of the present disclosure.

FIG. 51 is a graph showing the ELISA analysis showing binding of purified S-trimers to human ACE2 at various ACE2 concentrations according to an exemplary embodiment of the present disclosure.

FIG. 52 is a graph showing the decoration of phage T4 nanoparticles with spike ectodomain trimers via Spytag-SpyCatcher bridges according to an exemplary embodiment of the present disclosure.

FIG. 53 is a photo showing the In vitro assembly of S trimers on T4-SpyCatcher phage at increasing ratios of S-trimer molecules to Soc binding sites according to an exemplary embodiment of the present disclosure.

FIG. 54 is a representative cryo- EM image showing T4-SocA, T4-(Soc-SpyCatcher), and T4-(Soc-SpyCatcher)-S trimer according to an exemplary embodiment of the present disclosure.

FIG. 55 is a graph showing ELISA analysis of T4-S-trimer phage binding to ACE2 at various ACE2 concentrations according to an exemplary embodiment of the present disclosure.

FIG. 56 is a graph showing ELISA analysis of T4-S-trimer phage binding to ACE2 at various ACE2 concentrations according to an exemplary embodiment of the present disclosure.

FIG. 57 is a photo showing the binding of T4-S-trimer-GFP phage to HEK 293 cells expressing ACE2 according to an exemplary embodiment of the present disclosure.

FIG. 58 is a photo showing the expression of ACE2 on 293 cells according to an exemplary embodiment of the present disclosure.

FIG. 59 is a photo showing lack of binding of T4-GFP control phage (without S trimer) to ACE2-293 cells according to an exemplary embodiment of the present disclosure.

FIG. 60 is a photo showing a pipeline of generating SARS-CoV-2 vaccine candidates by sequential CRISPR engineering according to an exemplary embodiment of the present disclosure.

FIG. 61 is an image showing an example of phage sequential CRISPR engineering for creating “universal” SARS-CoV-2 vaccine according to an exemplary embodiment of the present disclosure.

FIG. 62 is a photo showing Western Blotting results confirming NP protein encapsidation in the phages containing CTSam- NP insertion at IPIII deletion site according to an exemplary embodiment of the present disclosure.

FIG. 63 is an image showing Balb/c mice immunized by intramuscular (i.m.) route using T4-SARS-CoV-2 vaccine formulations according to an exemplary embodiment of the present disclosure.

FIG. 64 is a photo showing formulations, groups and prime-boost immunization scheme used for mice vaccinations according to an exemplary embodiment of the present disclosure.

FIG. 65 is a graph showing the specific IgG antibody titers (endpoint) of anti-S-ecto of boost-2 sera (week 8 bleeding) from various groups assessed by ELISA. according to an exemplary embodiment of the present disclosure.

FIG. 66 is a graph showing the specific IgG antibody titers (endpoint) of anti-RBD of boost-2 sera (week 8 bleeding) from various groups assessed by ELISA according to an exemplary embodiment of the present disclosure.

FIG. 67 is a graph showing the specific IgG antibody titers (endpoint) of anti-NP of boost-2 sera (week 8 bleeding) from various groups assessed by ELISA according to an exemplary embodiment of the present disclosure.

FIG. 68 is a graph showing the specific IgG antibody titers (endpoint) of anti-E of boost-2 sera (week 8 bleeding) from various groups assessed by ELISA according to an exemplary embodiment of the present disclosure.

FIG. 69 is a graph showing the anti-S-ecto IgG1 antibody titers in the boost-2 sera (week 8 bleeding) from various groups according to an exemplary embodiment of the present disclosure.

FIG. 70 is a graph showing the anti-S-ecto IgG2a antibody titers in the boost-2 sera (week 8 bleeding) from various groups according to an exemplary embodiment of the present disclosure.

FIG. 71 is a graph showing the anti-RBD IgG1 antibody titers in the boost-2 sera (week 8 bleeding) from various groups according to an exemplary embodiment of the present disclosure.

FIG. 72 is a graph showing the anti-RBD IgG2a antibody titers in the boost-2 sera (week 8 bleeding) from various groups according to an exemplary embodiment of the present disclosure.

FIG. 73 is a graph showing the anti-NP IgG1 antibody titers in the boost-2 sera (week 8 bleeding) from various groups according to an exemplary embodiment of the present disclosure.

FIG. 74 is a graph showing the anti-NP IgG2a antibody titers in the boost-2 sera (week 8 bleeding) from various groups according to an exemplary embodiment of the present disclosure.

FIG. 75 is a graph showing the anti-E IgG1 antibody titers in the boost-2 sera (week 8 bleeding) from various groups according to an exemplary embodiment of the present disclosure.

FIG. 76 is a graph showing the anti-E IgG2a antibody titers in the boost-2 sera (week 8 bleeding) from various groups according to an exemplary embodiment of the present disclosure.

FIG. 77 is a graph showing the anti-RBD IgG antibody titers in the sera from group G5 (T4-HocA-SocΔ-S-ecto-Ee-NP) at 2 weeks (prime), 5 weeks (boost-1), and 8 weeks (boost-2) according to an exemplary embodiment of the present disclosure.

FIG. 78 is a graph showing the comparison of anti-RBD IgG antibody titers by ELISA using HEK293-produced RBD or E. coli-produced RBD as coating antigens in groups G3 (phage control), G7 (rRBD displayed T4), G8 (S trimer displayed T4), and G2 (S trimer& Alhydrogel) according to an exemplary embodiment of the present disclosure.

FIG. 79 is a graph showing the measurement of anti-S-ecto IgG antibody endpoint titers in sera from S-trimer & Alhydrogel (G2) group and T4-S-trimer (G8 and G9) groups at 2 weeks (prime), 5 weeks (boost-1), and 8 weeks (boost-2) according to an exemplary embodiment of the present disclosure.

FIG. 80 is a graph showing the comparison of anti-S-ecto IgG1 and IgG2a subtype antibody titers in sera from S-trimer & Alhydrogel (G2) group and T4-S-trimer (G8 and G9) groups at 8 weeks (boost-2) according to an exemplary embodiment of the present disclosure.

FIG. 81 is a graph showing the anti-S-ecto IgG1 antibody titers in the sera from 5-trimer & Alhydrogel (G2) group and T4-S-trimer (G8 and G9) groups at 2 weeks (prime), 5 weeks (boost-1), and 8 weeks (boost-2) according to an exemplary embodiment of the present disclosure.

FIG. 82 is a graph showing the anti-S-ecto IgG2a antibody titers in the sera from 5-trimer & Alhydrogel (G2) group and T4-S-trimer (G8 and G9) groups at 2 weeks (prime), 5 weeks (boost-1), and 8 weeks (boost-2) according to an exemplary embodiment of the present disclosure.

FIG. 83 is a graph showing the blocking of native RBD protein binding to 293-ACE2 by sera from phage control group (G3), S-trimer & Alhydrogel group (G2), and T4-S-trimer group (G8) at 500-fold serum dilution according to an exemplary embodiment of the present disclosure.

FIG. 84 is a graph showing the blocking of native RBD protein binding to 293-ACE2 by sera from phage control group (G3), S-trimer & Alhydrogel group (G2), and T4-S-trimer group (G8) at 2500-fold serum dilution according to an exemplary embodiment of the present disclosure.

FIG. 85 is a graph showing the neutralization antibody measurement according to an exemplary embodiment of the present disclosure.

FIG. 86 is a graph showing the percentage starting body weight of immunized mice at days post infection with 105 PFU SARS-CoV-2 MA10 according to an exemplary embodiment of the present disclosure.

FIG. 87 is a graph showing the survival of mice against SARS-CoV-2 MA10 challenge according to an exemplary embodiment of the present disclosure.

FIG. 88 is a graph showing the percentage starting body weight of immunized mice from groups G3 (phage control), G5 (T4-S DNA plus T4-S trimer protein), and G9 (T4-S trimer) at days post infection with 105 PFU SARS-CoV-2 MA10 according to an exemplary embodiment of the present disclosure.

FIG. 89 is a graph showing the survival rate of groups G3, G5, and G9 after virus challenge according to an exemplary embodiment of the present disclosure.

FIG. 90 is a graph showing the formulations, groups, and prime- boost immunization scheme for rabbit intramuscular vaccinations according to an exemplary embodiment of the present disclosure.

FIG. 91 is a graph showing the specific IgG antibody titers of anti-S-ecto in boost sera (10 days after boost) from groups G1 to G4 were assessed by ELISA according to an exemplary embodiment of the present disclosure.

FIG. 92 is a graph showing the specific IgG antibody titers of anti-RBD in boost sera (10 days after boost) from groups G1 to G4 were assessed by ELISA according to an exemplary embodiment of the present disclosure.

FIG. 93 is a graph showing the neutralization titers of serum from immunized rabbit according to an exemplary embodiment of the present disclosure.

FIG. 94 is a graph showing the comparison of NZW rabbit neutralization antibody titers in pre-immune and boost sera (10 days after boost) according to an exemplary embodiment of the present disclosure.

FIG. 95 is a graph showing the comparison of G1 (control phage) and G4 (Ee-NP-S trimer displayed phage) in anti-NP (E) IgG antibody titers. according to an exemplary embodiment of the present disclosure.

FIG. 96 is a graph showing the comparison of G1 (control phage) and G4 (Ee-NP-S trimer displayed phage) in anti-E IgG antibody titers. according to an exemplary embodiment of the present disclosure.

### DETAILED DESCRIPTION OF THE INVENTION

### Definitions

Where the definition of terms departs from the commonly used meaning of the term, applicant intends to utilize the definitions provided below, unless specifically indicated.

Unless defined otherwise, all technical and scientific terms used herein have the same meaning as is commonly understood to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

For purposes of the present disclosure, the term “comprising”, the term “having”, the term “including,” and variations of these words are intended to be open-ended and mean that there may be additional elements other than the listed elements.

For purposes of the present disclosure, directional terms such as “top,” “bottom,” “upper,” “lower,” “above,” “below,” “left,” “right,” “horizontal,” “vertical,” “up,” “down,” etc., are used merely for convenience in describing the various embodiments of the present disclosure. The embodiments of the present disclosure may be oriented in various ways. For example, the diagrams, apparatuses, etc., shown in the drawing figures may be flipped over, rotated by 90° in any direction, reversed, etc.

For purposes of the present disclosure, a value or property is “based” on a particular value, property, the satisfaction of a condition, or other factor, if that value is derived by performing a mathematical calculation or logical decision using that value, property or other factor.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

For purposes of the present invention, the term “capsid” and the term “capsid shell” refers to the protein shell of a virus comprising several structural subunits of proteins. The capsid encloses the nucleic acid core of the virus.

For purposes of the present invention, the term “nucleic acid” refers to polymers of nucleotides of any length, and include DNA and RNA. The nucleic acid bases that form nucleic acid molecules can be the bases A, C, G, T and U, as well as derivatives thereof. Derivatives of these bases are well known in the art. The term should be understood to include, as equivalents, analogs of either DNA or RNA made from nucleotide analogs. The term should also be understood to include both linear and circular DNA. The term as used herein also encompasses cDNA, that is complementary, or copy, DNA produced from an RNA template, for example by the action of reverse transcriptase.

For purposes of the present invention, the term “neck protein” and the term “tail protein” refers to proteins that are involved in the assembly of any part of the necks or tails of a virus particle, in particular bacteriophages. Tailed bacteriophages belong to the order Caudovirales and include three families: The Siphoviridae have long flexible tails and constitute the majority of the tailed viruses. Myoviridae have long rigid tails and are fully characterized by the tail sheath that contracts upon phage attachment to bacterial host. The smallest family of tailed viruses are podoviruses (phage with short, leg-like tails). For example, in T4 bacteriophage gp10 associates with gpl 1 to forms the tail pins of the baseplate. Tail-pin assembly is the first step of tail assembly. The tail of bacteriophage T4 consists of a contractile sheath surrounding a rigid tube and terminating in a multiprotein baseplate, to which the long and short tail fibers of the phage are attached. Once the heads are packaged with DNA, the proteins gp13, gp14 and gp15 assemble into a neck that seals of the packaged heads, with gp13 protein directly interacting with the portal protein gp20 following DNA packaging and gp14 and gp15 then assembling on the gp13 platform. Neck and tail proteins in T4 bacteriophage may include but are not limited to proteins gp6, gp25, gp53, gp8, gp10, gpl 1, gp7, gp29, gp27, gp5, gp28, gp12, gp9, gp48, gp54, gp3, gp18, gp19, gp13, gp14, gp15 and gp63.

For purposes of the present invention, the term “purified” refers to the component in a relatively pure state—e.g. at least about 90% pure, or at least about 95% pure or at least about 98% pure.

For purposes of the present invention, the term “immune response” refers to a specific response of the immune system of a biological specimen to antigen or immunogen. Immune response may include the production of antibodies and cellular immunity.

For purposes of the present invention, the term “immunity” refers to a state of resistance of a biological specimen to an infecting organism or substance. It will be understood that an infecting organism or substance is defined broadly and includes parasites, toxic substances, cancer cells and other cells as well as bacteria and viruses.

For purposes of the present invention, the term “immunization conditions” refers to factors that affect an immune response including the amount and kind of immunogen or adjuvant delivered to a biological specimen, method of delivery, number of inoculations, interval of inoculations, the type of biological specimen and its condition. “Vaccine” refers to pharmaceutical formulations able to induce immunity.

For purposes of the present invention, the term “bind,” the term “binding” and the term “bound” refers to any type of chemical or physical binding, which includes but is not limited to covalent binding, hydrogen binding, electrostatic binding, biological tethers, transmembrane attachment, cell surface attachment and expression.

For purposes of the present invention, the term “vector”, “vehicle”, and “nanoparticle” are used interchangeably. These terms refer to a virus or a hybrid viral particle that can be used to deliver genes or proteins.

For purposes of the present invention, the terms “efficiency of plating” and “EOP” are used interchangeably. These terms refer to a relative number of plaques that a phage stock is capable of producing. in the present disclosure, EOP is determined by dividing the pfu produced from infection of E. coli containing a spacer by the input pfu, in which the input pfu is the count of phages initially infect E. coli.

For purposes of the present invention, the term “reactogenic” refers to the property of a vaccine of being able to produce common, “expected” adverse reactions, especially excessive immunological responses and associated signs and symptoms, including fever and sore arm at the injection site. Other manifestations of reactogenicity typically identified in such trials include bruising, redness, induration, and swelling. The reactogenic effects of vaccine are often caused by adjuvant.

For purposes of the present invention, the term “codon optimization” refers to a process used to improve gene expression and increase the translational efficiency of a gene of interest by accommodating codon bias of the host organism.

For purposes of the present invention, the term “plaque” refers to clear zones formed in a lawn of cells due to lysis by phage. At a low multiplicity of infection (MOI) a cell is infected with a single phage and lysed, releasing progeny phage which can diffuse to neighboring cells and infect them, lysing these cells then infecting the neighboring cells and lysing them, etc, ultimately resulting in a circular area of cell lysis in a turbid lawn of cells.

For purposes of the present invention, the term “recombination frequency” refers to a measure of genetic linkage. Recombination frequency is the frequency, with which a single chromosomal crossover will take place between two genes. In the present disclosure, recombination frequency is determined by dividing the pfu when both plasmids are presented in the host cells by the pfu when only donor plasmid is presented in the host cells.

For purposes of the present invention, the terms “plaque-forming unit” and “pfu” are used interchangeably. These terms refer to a measure used in virology to describe the number of virus particles capable of forming plaques per unit volume. It is a proxy measurement rather than a measurement of the absolute quantity of particles.

For purposes of the present invention, the term “protective effecacy” refers to measured in a controlled test and is based on how many individuals who got vaccinated developed the ‘outcome of interest’ (usually disease) compared with how many individuals who got the placebo (dummy vaccine) developed the same outcome. In the present disclosure, the ‘outcome of interest’ include but not limited to weight loss of subject, such as an amimal, and death caused by a disease.

### Description

Genetic, biochemical, and structural studies on phage T4 including the recently developed CRISPR phage engineering constitute an extraordinary resource for creating universal vaccine development platform using T4. The atomic structures of all the capsid proteins including Soc, Hoc, as well as the entire capsid have been determined. It has also been demonstrated that Soc and Hoc can be used as efficient adapters to tether foreign proteins to T4 capsid. Both Soc and Hoc have nanomolar affinity and exquisite specificity, allowing up to 1,025 molecules of full-length proteins, domains, and peptides to be arrayed on capsid. T4 capsids so decorated with pathogen epitopes mimic PAMPs (pathogen-associated molecular patterns) of natural viruses and stimulate strong innate and as well as adaptive immune responses.

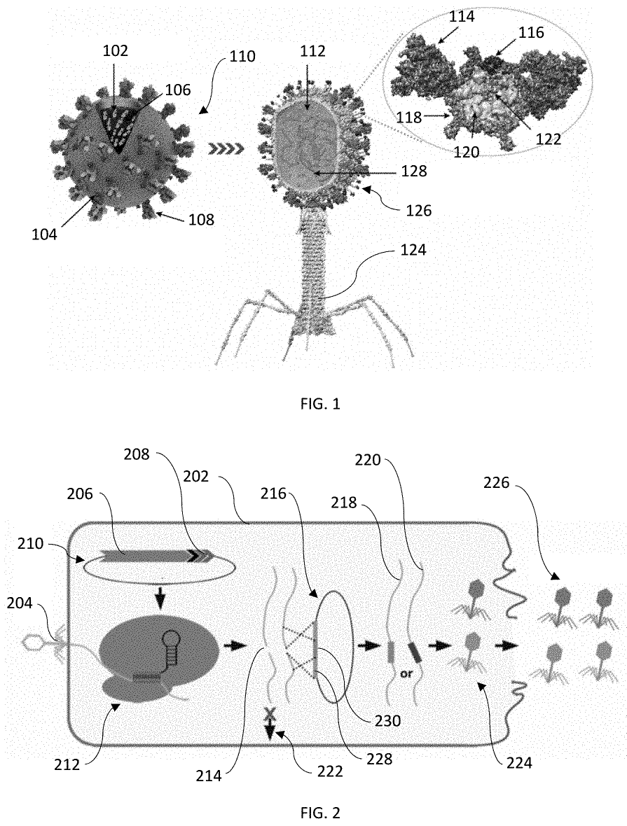

The large amount of nonessential genetic space available in T4 genome is usefule in developing a universal vaccine design template. SARS-CoV-2 (110) comprises nucleocapsid protein (NP) molecules (102), genome RNA (106), spike trimer (108) and envelope (E) epitopes (104), as shown in FIG. 1. Using SARS-CoV-2 as a model pathogen, a number of viral components, including spike (S), envelope (E), and nucleocapsid proteins (NP), can be inserted into phage by CRISPR engineering as DNA and/or protein. The T4 with insertion of SARS-CoV-2 viral components is illustrated in FIG. 1. These were re-combined to create phages in desired target combinations by simple phage infections. Thus, a pipeline of vaccine candidates in dozens of combinations can be generated, demonstrating the unprecedented engineering power and flexibility of this approach.

In one embodiment, A number of SARS-CoV-2 components were incorporated into T4 phage by CRISPR engineering. The design was such that each of these components occupied an appropriate compartment in the phage nanoparticle as shown in FIG. 1. For instance, mammalian-expressible spike/RBD gene(s) (128) as part of packaged genome, spike trimer (114) and envelope (E) epitopes (116) as surface decorations, and nucleocapsid protein (NP) molecules (112) as capsid core co-packaged with genome.

In one embodiment, when tested in mouse and New Zealand White rabbit models, T4 phage decorated with S-trimers elicited robust ACE-2 receptor blocking and virus neutralization established a new bacteriophage nanovaccine framework for rapid and multiplex design of effective vaccine candidates potentially against any emerging pathogen in the future.

Construction of T4-SARS-CoV-2 recombinant phages by CRISPR engineering

In one embodiment, a series of CRISPR-E. coli strains were constructed to insert SARS-CoV-2 gene segments into T4 phage genome. FIG. 2 shows the schematic of T4 CRISPR engineering usingE. coli. As shown in FIG. 2, each CRISPR-E. coli strain (202) harbored two plasmids: one “CRISPR” plasmid (210) a gene (206) expressing the genome editing nuclease, either type II Cas9 or type V Cpf1 nuclease, and CRISPR RNAs (crRNAs or “spacer” RNAs) (208) corresponding to target protospacer sequence(s) in phage genome, and a second “donor” plasmid (216) containing a donor sequence (230). In a preferred embodiment, the donor sequence (230) SARS-CoV-2 sequence. The latter also has ˜500 bp homologous flanking arms (228) of phage genome corresponding to the point of insertion. Also shown in FIG. 2, after a wild-type T4 (204) infects the CRISPR-E. coli strains, the genome of the wild-type T4 is cut by CRISPR-Cas9/Cpf1 produced as a result of expression of genes in the “CRISPR” plasmid to form a double-strand break (DSB) (214). The DNA strand with a break but does not receive a donor sequence (230) from the donor plasmid (216) will result in no production of new phages (222). After sequence exchange with the donor plasmid (216), either a DNA sequence with insertion (218) or a DNA sequence with deletion (220) will be produced. The engineered DNA sequences with either insertion (218) or delition (220) are packed into recombinant T4 (226), which are then released (224) from CRISPR-E. coli strains.

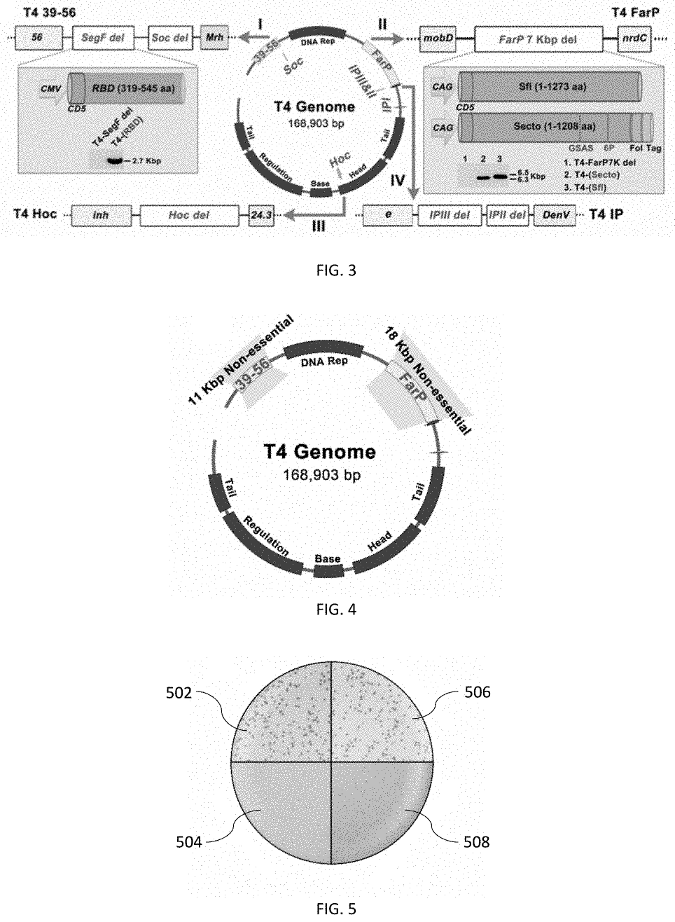

In one embodiment, four nonessential regions of the T4 genome were chosen for insertion of various SARS- CoV-2 genes, as illustrated in FIG. 3. When these E. coli were infected by T4 as shown in FIG. 2, a double-stranded break (214) would occur in the protospacer sequence by Cas9 or Cpf1 that inactivated the phage genome and no phage (222) could be produced. However, the highly recombinogenic T4 phage allowed efficient recombination between the cleaved DNA and the donor plasmid (216) through the flanking homologous arms (228), transferring the CoV-2 gene (230) into phage genome and propagated it as part of phage infection. The same strategy was used to introduce many other genetic modifications including deletions, by simply creating that modification in the donor plasmid.

In one embodiment, the recombinant phages were constructed by deleting certain known “nonessential” segments of the phage genome. The “nonessential” segments deleted included about 18 kb FarP, about 11-kb 39-56, or both (about 29 kb), that created space for CoV-2 insertions. The locations of segment FarP and segment 39-56 on T4 genome are illustrated in FIG. 4. These deletions can impact the size of plaque formed by the phages. FIG. 5 shows plaque size of wild-type (WT) (502), T4-FarP 18 kb del. (506), T4-39-56 11 kb del. (504), and T4-FarP&39-56 29 kb del. (508) phages. As shown in FIG. 5, T4-39-56 11 kb del. (504), and T4-FarP&39-56 29 kb del. (508) phages formed smaller plaque compared to WT phages. However, the yields of these phages were low, about 1-2 orders of magnitude lower than the wild-type (WT) phage.

In another embodiment, the recombinant phages were constructed by deleting shorter segments, since yield is critical for vaccine manufacture. The shorter segments deleted included about 675 bp SegF within 39-56 and about 7 kb segment within FarP. The structure of T4 genome is illustrated in FIG. 3. The location and structure of FarP, 39-56, SegF and the 7 kb segment within FarP are also illustrated in segments I and I in FIG. 3. The yields of these phages, which is named 7de1. SegFdel.T4, were similar to the WT phage, suitable for SARS-CoV-2 vaccine design. In FIG. 3, “6P” refers to six proline substitutions in S-ecto, including F817P, A892P, A899P, A942P, K986P, and V987P; “Fol” refers to T4 fibritin motif Foldon for efficient trimerization; “Tag” refers to octa-histidine and twin-strep tags; and Furin cleavage site RRAR was mutated to GSAS to stabilize trimers in a prefusion state.

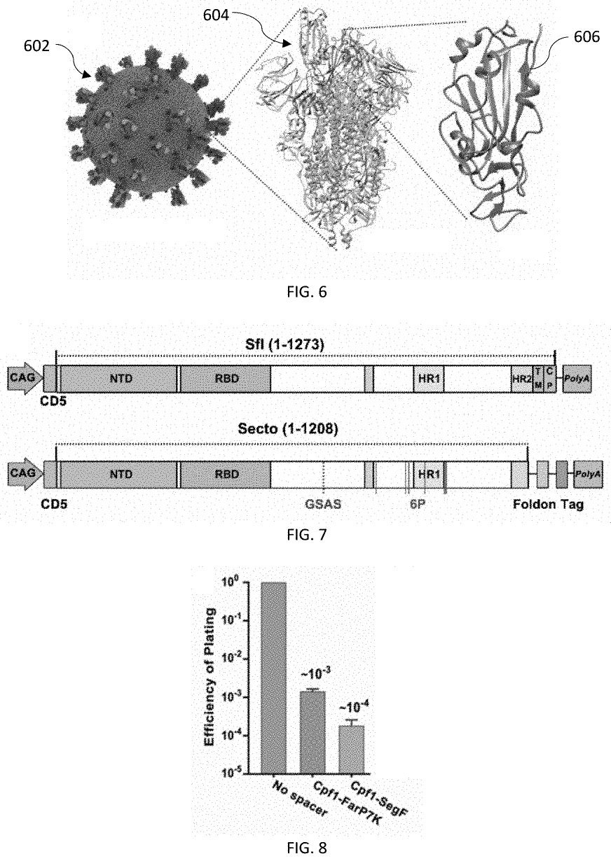

In one embodiment, three SARS-CoV-2 spike gene variants corresponding to i) 1273 aa WT full-length (S-fl), ii) 1208 aa ectodomain (S-ecto, aa 1-1208), and iii) 227 aa receptor binding domain (RBD, aa 319-545) were engineered as expressible cassettes and inserted into 7del.SegFdel. T4. The three spike gene variants and their respective location of insertion are illustrated in FIG. 3. FIG. 6 illustrates the schematic of SARS-CoV-2 virus, spike trimer, and receptor binding domain (RBD). As shown in FIG. 6, RBD is a portion of Strimer.

In one embodiment, the spike gene variants were codon-optimized and kept under the control of a strong mammalian expression promoter, either CMV or CAG, and a human CD5 signal peptide fused to the N-terminus for efficient secretion. The S-full length (S-fl) and S-ectodomain (S-ecto) expression cassettes used for insertion into T4 genome are illustrated in FIG. 7. The location of promoter and CD5 signal peptide are illustrated in segments I and II of FIG. 3. Also as shown in FIG. 3, the 5-ectodomain recombinant contained additional mutations including six proline substitutions that imparted greater stability and about 10-fold greater expression, as was described by Hsieh et al.¹

In one embodiment, CRISPR E. coli cells containing the Cas9/Cpf1-spacer plasmid but lacking the spike gene donor plasmids was created as a control. The control CRISPR E. coli cells gave very few or no plaques when infected with 7del.SegFdel phage. FIG. 8 shows EOP of representative Cpf1-FarP7K and Cpf1-SegF spacers, including Cpf1-FarP7K-spl, Cpf1-FarP7K-sp2, Cpf1-SegF-spl and Cpf1-SegF-sp2. EOP was determined by dividing the pfu produced from infection of E. coli containing a spacer by the input pfu. The E. coli cells used to conduct experient shown in FIG. 8 does not contain any donor plasmind. Either Cpf1-FarP7K or Cpf1-SegF spacers in E. coli cells cuts the invading phage genome, leading to a reduced number of plaques formed. Thus, when there is no spacer in E. coli cells, none of the invading phages will have a cut in their gemons, resulting in no reduction in plague formation. Therefore, the input pfu equal to the pfu produced from infection of E. coli containing no spacer, Cpf1-FarP7K spacer and Cpf1-SegF spacer from left to right in FIG. 8. According to FIG. 8, the EOPs of Cpf1-FarP7K and Cpf1-SegF spacers are no greater than aobut 10⁻³ and 10⁻⁴, respectively.

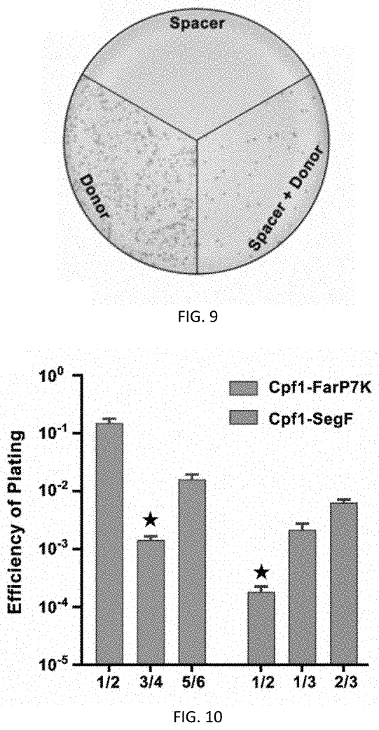

In one embodiment, the phage plaques formed by phages obtained from phage infection of bacteria containing Cpf1-FarP7K spacer only, S-ecto donor only, or Cpf1-FarP7K spacer combined with S-ecto donor were compared. As shown in FIG. 9, no phage plaques formed when the bacteria contained Cpf1-FarP7K spacer only, while there are phage plaques formed when he bacteria contained both Cpf1-FarP7K spacer and S-ecto donor although the number of phage plaques formed was not as many as that when only S-ecto donor was presented in the bacteria.

In one embodiment, the EOP of different Cpf1-FarP7K and Cpf1-SegF spacers were different. As shown in FIG. 10, among the three sets of Cpf1-FarP7K spacers and three sets of Cpf1-SegF spacers tested, Cpf1-FarP7K spacer 3/4 and Cpf1-SegF spacer 1/2 have the lowest EOP.

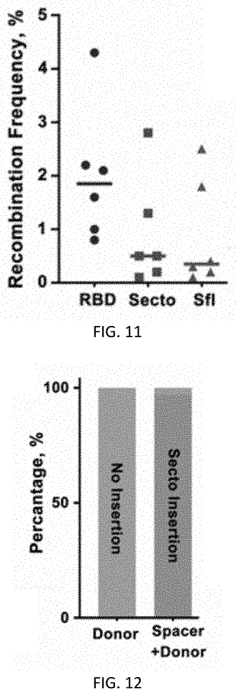

In one embodiment, the recombination frequency of using the CRISPR E. coli cells containing both the Cas9/Cpf1-spacer plasmid and donor plasmid is upto about 4.5%. The recombination frequency is the pfu when both plasmids are presented in the CRISPR E. coli cells divided by the pfu when only donor plasmid is presented in the CRISPR E. coli cells. FIG. 11 shows the recombination frequencies of inserting three spike genes, including RBD, S-ecto, and S-fl. As shown in FIG. 11, the recombination frequencies of inserting different genes are different, with the recombination frequency of RBD insertion the highest.

In one embodiment, at least 95% of recombinant T4 phages contained the correct insertion of spike genes. FIG. 12 shows the result of insertion after DNA sequencing of thirty independent plaques, confirming that >95% of the plaques generated in S-ecto recombination contained the correct S-ecto insert.

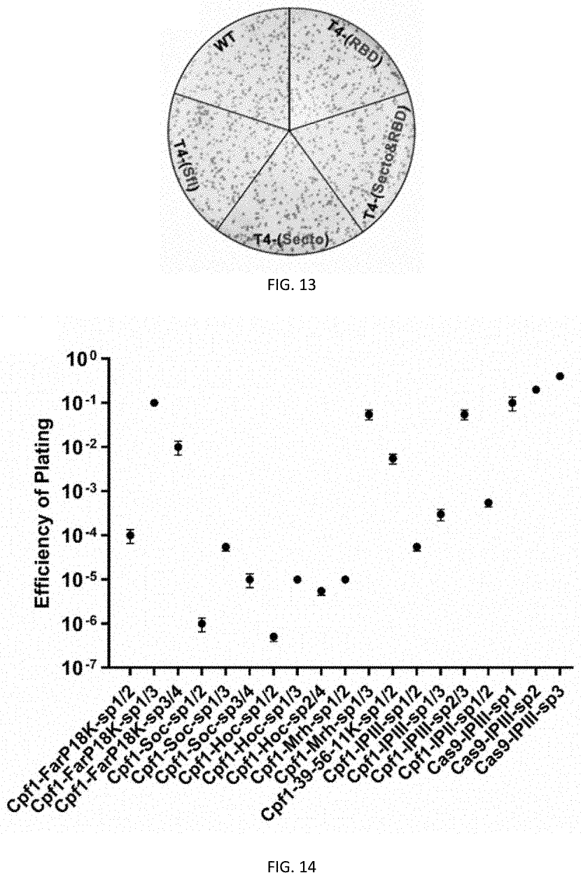

In one embodiment, the phages with an insertion of spike genes have similar plaque forming ability as the WT phage. FIG. 13 shows the plaque size of wild-type (WT) phages, T4-RBD, T4-S-fl, T4-S-ecto, and T4-(S-ecto)-RBD recombinant phages, confirming similar plaque size.

In one embodiment, a similar CRISPR strategy as described above was used for creating deletions and/or insertions at the other sites, indlucing IPIII, IPII, Hoc, and Soc. The location and structure of IPIII, IPII, Hoc, and Soc are illustrated in segments III and IV of FIG. 3. The EOP of IPIII, IPII, Hoc, and Soc insertions using different spacers are shown in FIG. 14. Simmilar to the results of Cpf1-FarP7K and Cpf1-SegF, the EOP varied at different sites and by using different spacers.

### Encapsidation of SARS-CoV-2 Nucleocapsid Protein (NP)

SARS-CoV-2 infected patients have been reported to generate robust NP-specific immune responses including cytotoxic T cells that might be important for protection and virus clearance².

In one embodiment, NP was incorporated into the T4 nanoparticle by designing a CRISPR strategy that packaged NP protein molecules inside the phage capsid along with the genome. As NP is a nucleic acid binding protein, the packaged phage genome might provide an appropriate environment to localize this protein.

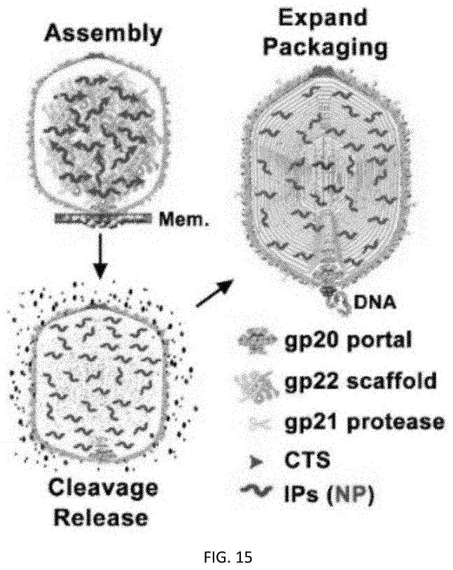

During T4 phage morphogenesis, the major capsid protein gp23 assembles around a scaffolding core formed by a cluster of proteins including three nonessential, highly basic, “internal proteins”; IPI, IPII, and IPIII. Following assembly, most of these scaffold proteins are degraded to small peptides and expelled from the capsid creating space for genome packaging. This assembly process is illustrated in FIG. 15. The IPs, however, are cleaved only once, next to a ˜10 aa N-terminal capsid targeting sequence (CTS) (MKTYQEFIAE). While the CTS leaves the capsid, ˜1,000 molecules of IPs remain inside the “expanded” capsid and presumably are involved in host takeover to overcome host defense system³.

Previous studies showed that when the IPs are replaced with foreign proteins fused to N-terminal CTS, the foreign proteins are targeted to the core and remain in the capsid interior following CTS removal⁴.

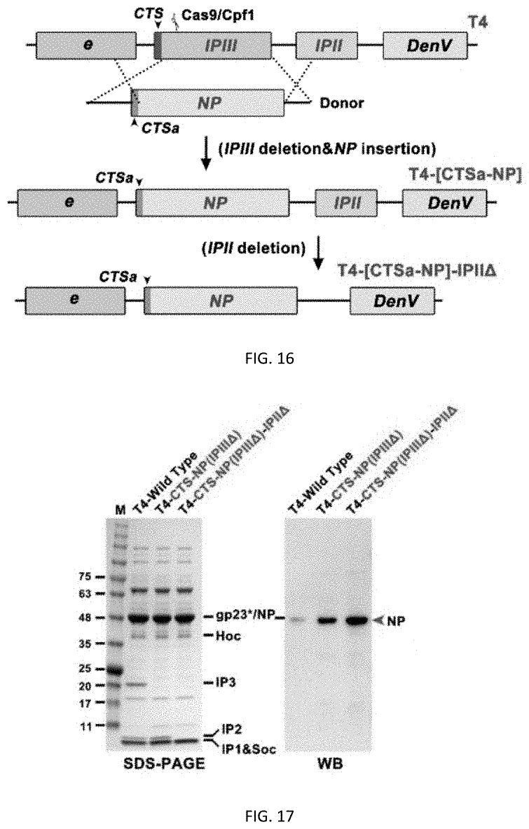

In one embodiment, IPs were replaced with CoV-2 NP. FIG. 16 illustrates the steps of the replacement of Ips with CoV-2 NP. Briefly, to replace IPs with CoV-2 NP, an IPIII deletion phage was first created using appropriate spacer and donor. Then, a CTS-NP fusion sequence was transferred into this phage by inserting a CTS-fused, codon optimized, SARS-CoV-2 NP gene kept under the control of the native IPIII promoter into the IPIII del. phage. Next, IPII was deleted to reduce protein packaging competition and increase the copy number of NP. The successful deletion of IPIII and IPII as well as the insertion of NP have been confirmed by SDS-PAGE and Western Blotting analysis of phage particles, as shown in FIG. 17. The left panel of FIG. 17 is the SDS-PAGE, in which the lack of IP3 band in the T4-CTS-NP(IPIIΔ) and T4-CTS-NP(IPIIΔ)-IPIIΔ columns and the lack of IP2 band in the T4-CTS-NP(IPIIΔ)-IPIIA column confirm the deletion of IPIII and IPII. On the other hand, the right panel of FIG. 17 is Western Blotting results, in which the presence of a significantly heavier NP band in both T4-CTS-NP(IPIIΔ) and T4-CTS-NP(IPIIΔ)-IPIIA columns compared to the T4-Wild Type column confirmed the insertion of NP.

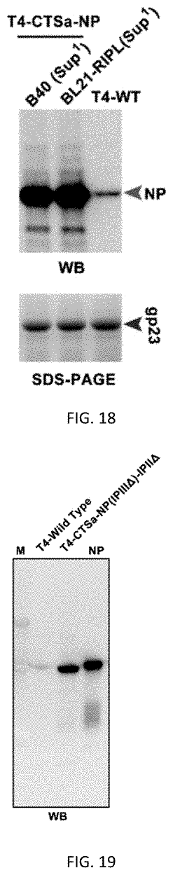

In one embodiment, an amber mutation was introduced into the CTS sequence, changing the TTT corresponding to Phe at site aa 7 to TAG corresponding to amber, because, for unknown reasons, the donor plasmid containing the WT CTS sequence was found to be toxic to E. coli. This CTSa-NP phage when grown on amber suppressor E. coli B40 (Sup1) or BL21-RIPL (Sup1) expressed the nucleocapsid protein and encapsidated it as demonstrated by SDS-PAGE shown in FIG. 18 with NP-specific monoclonal Abs and Western Blotting using A second NP-specific monoclonal antibody shown in FIG. 19.

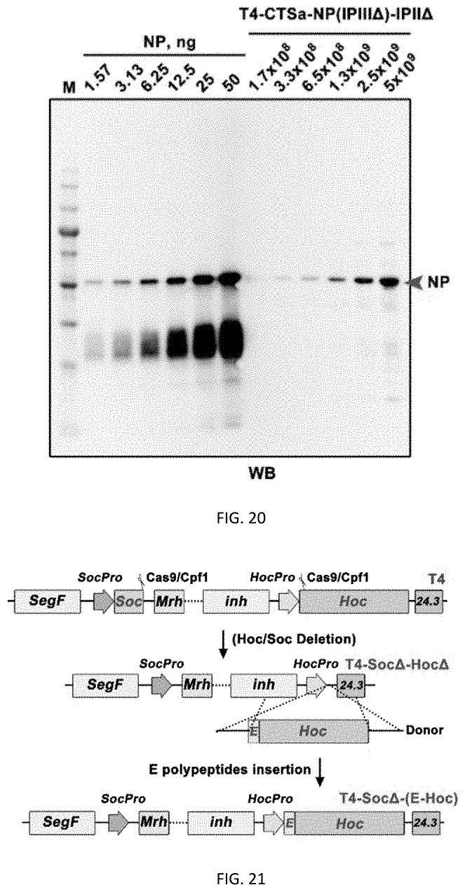

In one embodiment, the copy number of NP is about 70 NP molecules per phage capsid. FIG. 20 shows quantification of the amount of NP protein and the corresponding number of T4-CTS-NP(IPIIΔ)-IPIIA. The molecular weight of NP is about 46 kDa. The amount (ng) of NP was quatified by comparing the band density of sample with NP standards shown in FIG. 20. The NP copy number per phage was calculated based on the quantification of NP and T4 phages obtained from FIG. 20. The amount of NP (ng) on the top of FIG. 20 is approximately the amount of the NP standards, which is close to but may not be the exact amount of NP in samples.

### Display of SARS CoV-2 Epitopes on T4 Phage

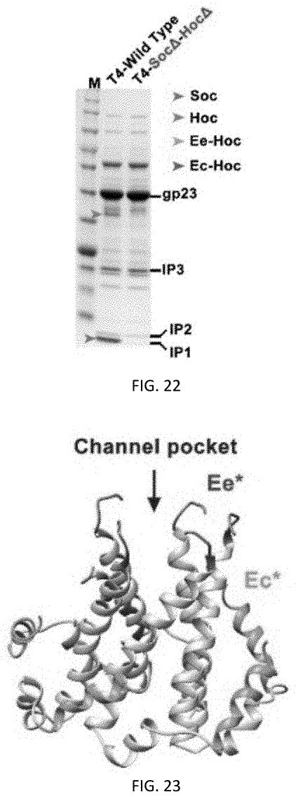

In one embodiment, SARS-CoV-2 antigens were incorporated onto the nanoparticle surface. In order to incorporate SARS-CoV-2 antigens on the surface of T4, Hoc and Soc genes were deleted from each of the above spike & NP phage genomes to create T4-SocΔ-HocA phages and then Hoc- and Soc-fused CoV-2 genes under the control of their respective native promoters were inserted. In one preferred embodiment, the inserted Hoc-fused CoV-2 gene encodes E epitope and the resulting recombinant T4 phages are T4-SocΔ-(E epitope-Hoc) phages. The inserted Hoc- and Soc-fused CoV-2 genes, upon phage infection, would express and assemble the epitopes encoded by the inserted CoV-2 genes on T4 capsid surface. The steps of constructing T4-SocΔ-HocA and T4-SocΔ-(E epitope-Hoc) phages are illustrated in FIG. 21. The successful deletion of Hoc and Soc was confirmed by SDS-PAGE, which is shown in FIG. 22.

In one embodiment, the inserted E epitope was constructed by fusing the gene segments corresponding to the N-terminal 12-aa ectodomain peptide (Ee) or the 18-aa peptide from the C-terminal region (Ec) of CoV-2 envelope (E) protein fused to the N-terminus of Hoc. The pentamieric structure of CoV-2 envelope (E) protein and the Ee and Ec domains are illustrated in FIG. 23. The N-terminal seven residues and C-terminal ten residues are not shown in FIG. 23 due to the lack of a corresponding segment in the structural template used for homology modeling. In FIG. 23, Ee* indicates amino acid (aa) 8-12 and Ec* indicates aa 53-65.

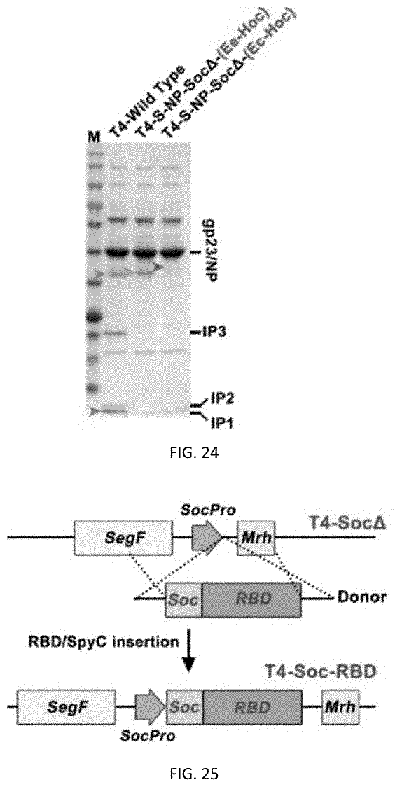

These peptides are predicted to be exposed on the SARS-CoV-2 virion and shown to elicit T cell immune responses in humans⁵. In one embodiment, by virtue of fusion to the N-terminus of Hoc, these epitopes would be exposed at the tip of the ˜170 Å-long Hoc fiber. The Ee and Ec recombinant phages indeed showed an upward shift of the Hoc band upon SDS-PAGE, consistent with the increase of mass of the fused peptides. The SDS-PAGE is shown in FIG. 24, in which the shift of the Hoc band is identified by the arrows pointing to the corresponding bands in the T4-S-NP-SocΔ-(Ee-Hoc) and T4-S-NP-SocΔ-(Ec-Hoc) columns.

In one embodiment, the 12-aa Ee peptide was displayed at the maximum copy number, up to about 155 copies per capsid, without significantly affecting phage yield.

In one embodiment, the 18-aa Ec peptide showed lower epitope copies, as indicated by the fade Hoc band in the T4-S-NP-SocΔ-(Ec-Hoc) column of FIG. 24.

### Display of SARS-CoV-2 Receptor Binding Domain on T4 Phage

In one embodiment, CoV-2 receptor binding domain (RBD) was displayed on the capsid surface as a Soc-fusion, using a similar strategy as described above. FIG. 25 illustrates the steps of inserting Soc-RBD gene into phage genome at the Soc deletion site.

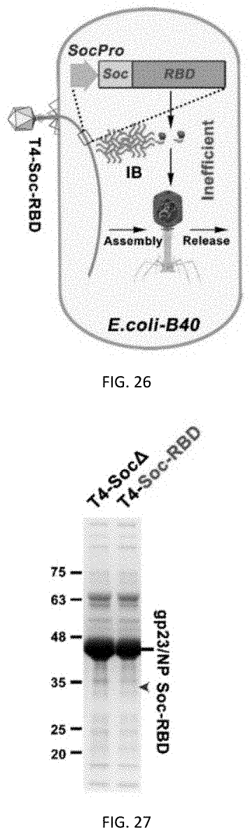

In one embodiment, the copy number of the displayed RBD was very low, due to inefficient in vivo display of E. coli-expressed Soc-RBD on T4 phage. FIG. 26 schematically shows the inefficient in vivo display of E. coli-expressed Soc-RBD on T4 phage. The inefficient in vivo display of E. coli-expressed Soc-RBD on T4 phage was also confirmed by SDS-PAGE resultes, ass shown in FIG. 27. In FIG. 27, the Soc-RBD band is much lighter in the T4-Soc-RBD column than that in the T4-SocΔ column.

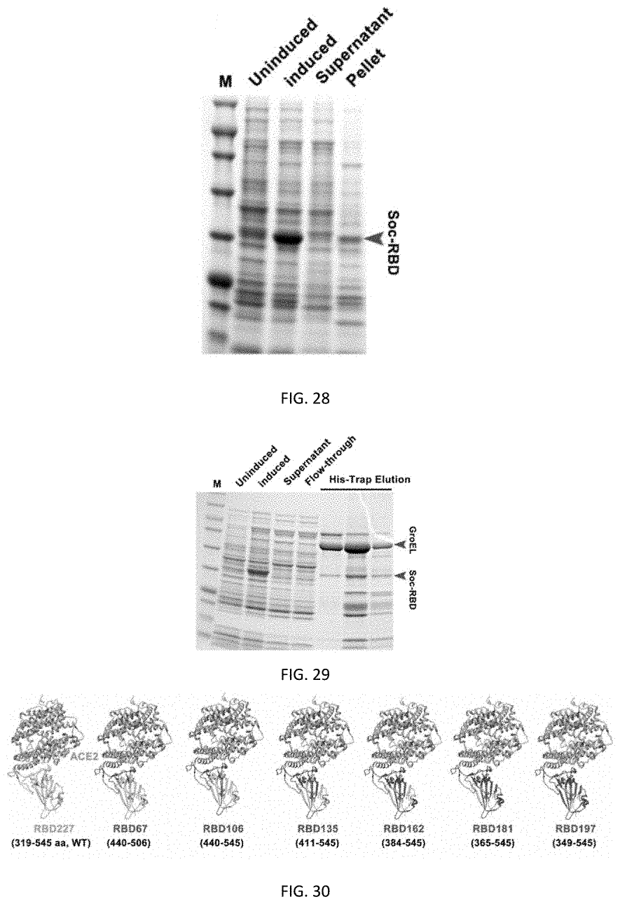

RBD contains ˜82.5% non-hydrophilic residues. In one embodiment, RBD formed insoluble inclusion bodies when expressed from a strong promoter. The insolubility of RBD was confirmed in the solubility analysis shown in FIG. 28. In FIG. 28, the presence of Soc-RBD in the pellet and absence in the supernatant of E. coli lysate indicates insolubility. The insolubility of RBD was also confirmed by recovering RBD from supernatant, with the results shown in FIG. 29. As shown in FIG. 29, very little soluble Soc-RBD was recovered after concentration of the any soluble Soc-RBD by purification on a HisTrap Ni affinity column. No significant Soc-RBD was detected in supernatant and flow-through, and very little Soc-RBD was eluted along with E. coli GroEL chaperone.

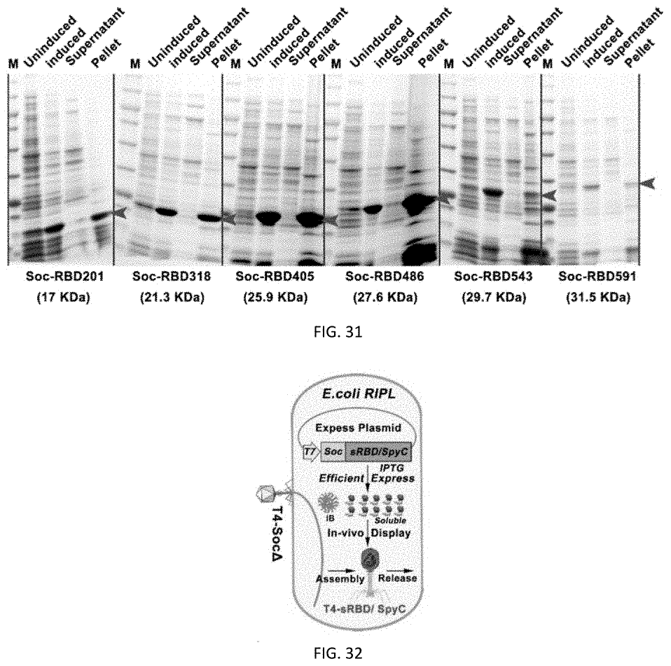

In one embodiment, numerous N- and C-terminal truncations of RBD, one of which is the shortest receptor binding motif of 67 aa, were constructed, however none showed a significant improvement in solubility and copy number (FIG. S4B). The structures of these truncated RBD are shown in FIG. 30. In FIG. 30, the RBDs bind to human ACE2. The Protein Data Bank (PDB) code for the SARS-CoV-2 RBD-ACE2 complex is 6M0J6. The truncated RBDs were generated using Chimera software. The solubility analysis of Soc-fused truncated RBDs is shown in FIG. 31. After lysis of E. coli and centrifugation, the supernatant and the pellet were analyzed by SDS-PAGE, with the results shown in FIG. 31. The presence of Soc-truncated RBDs in the pellet and their absence in the supernatant demonstrated insolubility. The arrowhead in FIG. 31 indicates the band position of various Soc-truncated RBDs. Therefore, alternative strategies were resorted to display RBD on the phage capsid.

In one embodiment, E. coli expressing Soc-RBD from a plasmid under the control of the phage T7 promoter constructed. Under the control of the phage T7 promoter, the pre- expression of Soc-RBD can be kept at low level.

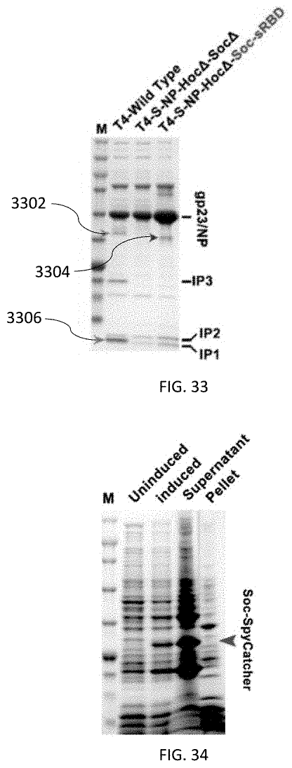

In one embodiment, a low level pre-expression of Soc-RBD would keep it in soluble form that could then assemble on capsids produced during phage infection. FIG. 32 schematically shows Soc-sRBD or Soc-SpyCatcher (SpyC) in vivo display on T4-SocΔ capsid. As shown in FIG. 32, Soc-sRBD or Soc-SpyCatcher expression under the control of phage T7 promoter was induced by IPTG. Most of the expressed Soc-RBD was in the inclusion body (IB). Soluble Soc-sRBD (minor amount) or Soc-SpyC can be efficiently displayed on capsid. As shown in FIG. 33, which is the SDS-PAGE resuts, phage isolated from E. coli expressing Soc-RBD from a plasmid under the control of the phage T7 promoter showed improved display, about 100 copies of RBD (sRBD) per phage particle. The band of Hoc (3302) and Soc (3306) in the column T4-Wild Type and band of Soc-sRBD (3304) are shown in FIG. 33.

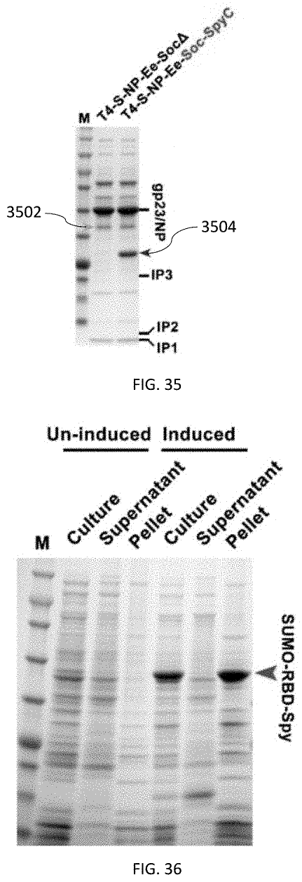

In one embodiment, the well-established spytag-spycatcher technology was deployed to display RBD on T4 phage. The optimized spycatcher and spytag from Streptococcus pyogenes, interact with eath other at least picomole affinity, indicating approaching “infinite” affinity with second-order rate constant: 5.5×10⁵M⁻¹ s⁻¹, and exquisite specificity that then leads to a covalent bond formation⁷. In one embodiment, to display RBD, phage decorated with the ˜12.6 kDa soluble spycatcher was produced by growing the T4-Spike-Ee-NP-SocΔ phage on E. coli expressing Soc-spycatcher fusion protein from the T7 expression plasmid. The expression of the Soc-spycatcher fusion protein is illustrated in FIG. 32. The improvement in solubility of Soc-spycatcher has been confirmed by solubility analysis of Soc-SpyCatcher shown in FIG. 34. As shown in FIG. 34, Soc-SpyCatcher expression was driven by the phage T7 promoter and most of the expressed protein remained in the supernatant indicates its high solubility. Phage prepared from these infections contained ˜300-600 copies of Soc-spycatcher per capsid, as shown in FIG. 35. FIG. 35 shows the Ee-Hoc band (3502) and Soc-SpyC band (3504). In determining the copy number of Soc-spycatcher per capsid, the SDS-PAGE band intensity of Soc-spycatcher was compared with that of major capsid protein gp23. Then the Soc-spycatcher copy number was determined based on 930 copies of gp23 per capsid. In FIG. 35, the gp23 and NP bands overlap, because gp23 and NP have very similar molecular weight.

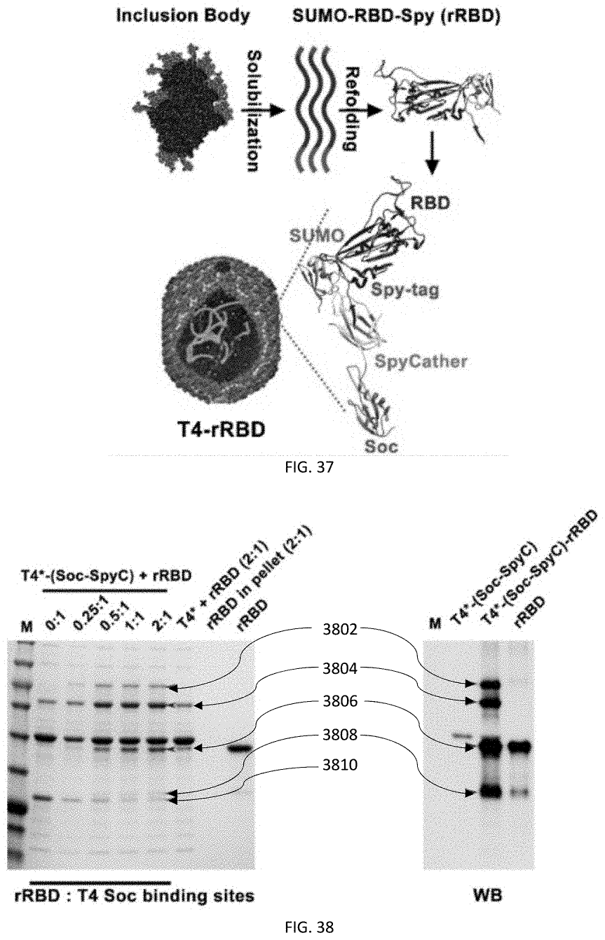

In another embodiment, RBD was expressed as Sumo-RBD-Spytag fusion protein in E. coli. The Sumo domain was supposed to enhance the expression and solubility of RBD but it resulted in only a small improvement, as shown in FIG. 36, which is the solubility analysis of SUMO-RBD-Spytag. Therefore, the Sumo-RBD-Spytag protein was purified from insoluble inclusion bodies by urea denaturation and refolding, forming rRBD, which was displayed in vitro on the spycatcher phage. The steps of purifying the Sumo-RBD-Spytag protein and in vitro display are illustrated in FIG. 37. Using the method illustrated in FIG. 37, refolded SUMO-RBD-Spytag (rRBD) protein molecules were efficiently displayed on T4-SpyCatcher phage surface via Spytag-SpyCatcher bridging. As shown in FIG. 38, SDS-PAGE and WB of the phage particles showed that the Sumo-RBD-Spytag was efficiently captured by the spycatcher phage as shown by the disappearance of the spycatcher band and appearance of higher molecular weight band(s). To determine the display of rRBD on the T4-SpyCacher surface at different ratios of rRBD molecules to capsid Soc binding sites ranging from 0:1 to 2:1, phage and rRBD were incubated at 4° C. for 1 hr, followed by centrifugation. The pellet was suspended for SDS-PAGE. RBD specific antibody was used to verify the displayed rRBD and rRBD-SpyCatcher-Soc complexes. In FIG. 38, T4* indicates T4-S-ecto-NP-Ec-SocA recombinant phage. The purified rRBD shows two bands on SDS-PAGE, with the band corresponding to a smaller molecular weight the truncated rRBD. Both full length and truncated rRBD contain spytag and can interact with spycatcher on T4, forming two conjugated proteins with higher molecular weight. Therefore, four rRBD-containing bands appeared after phage display of rRBD. FIG. 38 shows each of these bands, including conjugated full-length rRBD (3802), conjugated truncated rRBD (3804), unconjugated full-length rRBD (3806), unconjugated truncated rRBD (3808) and Soc-SpyCatcher (3810), respectively. According to FIG. 38, saturation was reached at a relatively low ratio of 2:1 of Sumo-RBD-Spytag to Spycatcher phage, consistent with the high affinity interaction between these two components. The copy number was about 300 rRBD molecules per capsid, also according to FIG. 38.

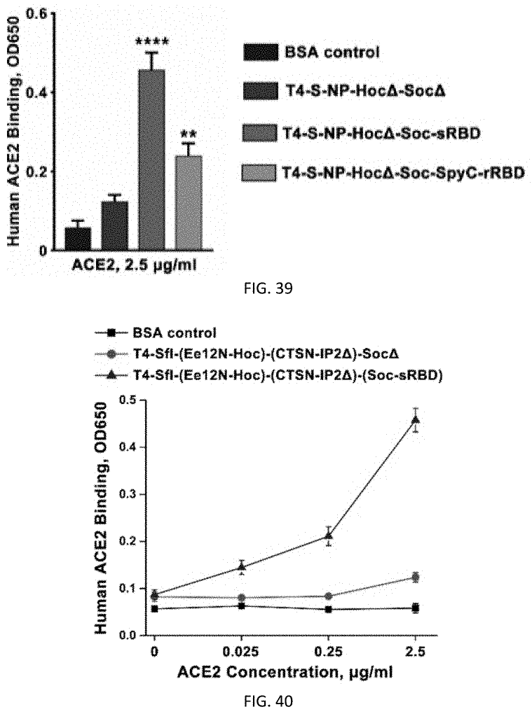

In one embodiment, the sRBD and rRBD phages produced as above, such as T4-Spike-Ee-CTSam-NP-sRBD and T4-Spike-Ee-CTSam-NP-rRBD, bound to human ACE2 receptor protein. FIG. 39 shows the comparison of binding of RBD phages to soluble human ACE2 receptor, when the concentration of ACE2 is 2.5 μg/ml. FIG. 40 shows comparison of binding of RBD phages to soluble human ACE2 receptor at different concentrations of ACE2.

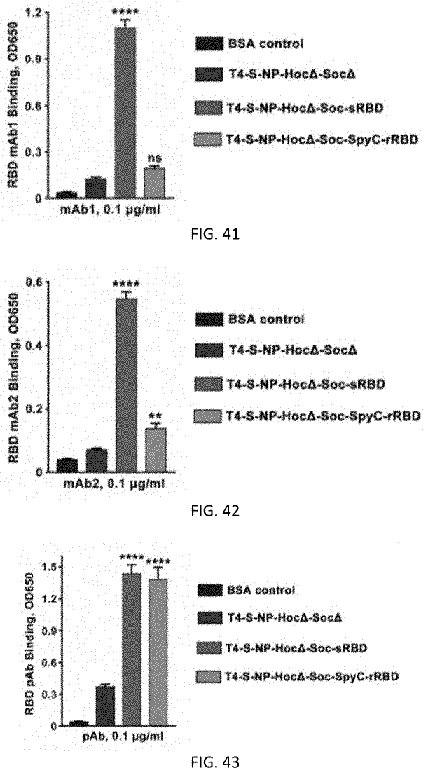

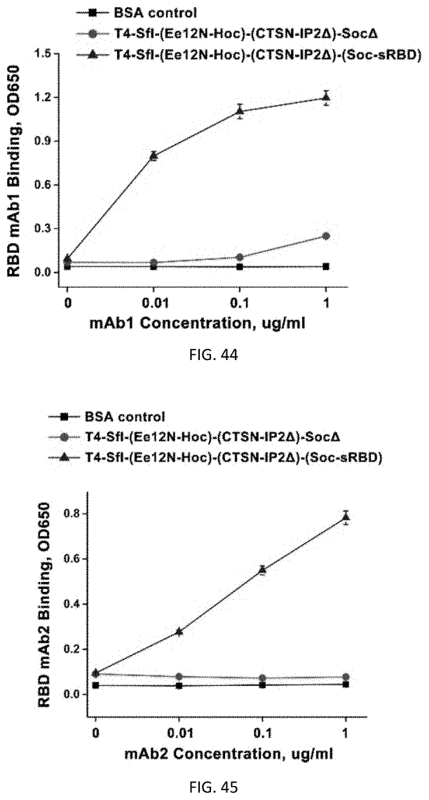

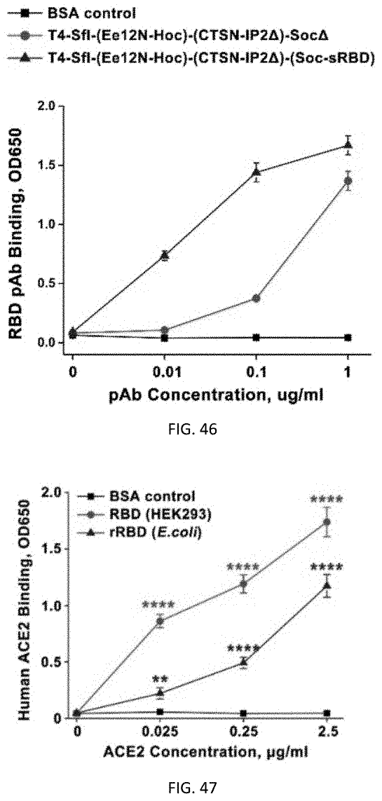

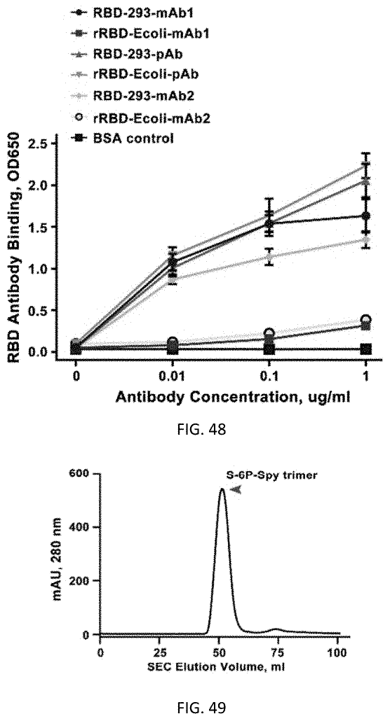

In one embodiment, rRBD phages also bind to some of the RBD-specific monoclonal antibodies (mAbs) and polyclonal antibodies (pAbs) but not to all, as shown in FIGS. 41-46. However, these RBDs exhibited considerably lower ACE2- or antibody-binding ability when compared to mammalian-expressed RBD. These data suggest that the E. coli-produced RBDs are not properly folded, which is also consistent with the co-purification of a 65-kDa E. coli GroEL chaperone that indicated the presence of partially folded and/or misfolded protein. FIGS. 47-48 compare the ACE2 receptor and antibody binding to RBD expressed in human HEK293 cells and E. coli. In FIG. 47, ** refers to P<0.01, **** refers to P<0.0001 and “ns” (no significance) refers to P>0.05. In FIG. 48, the 293-RBD showed much greater binding to mAbl and mAb2 than the E. coli rRBD, while binding to pAbs was similar.

Decoration of Phage T4 Nanoparticles with Spike Ectodomain Trimers

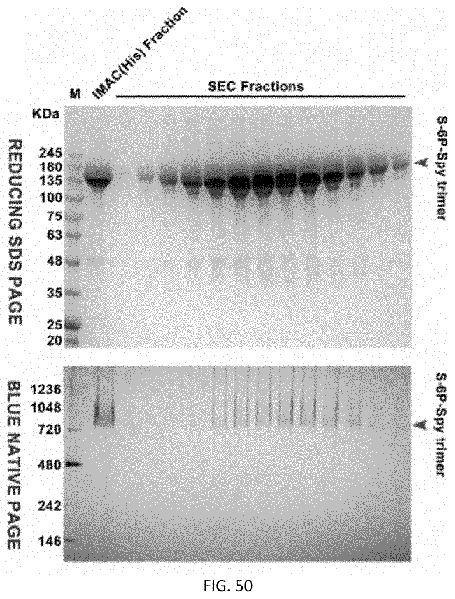

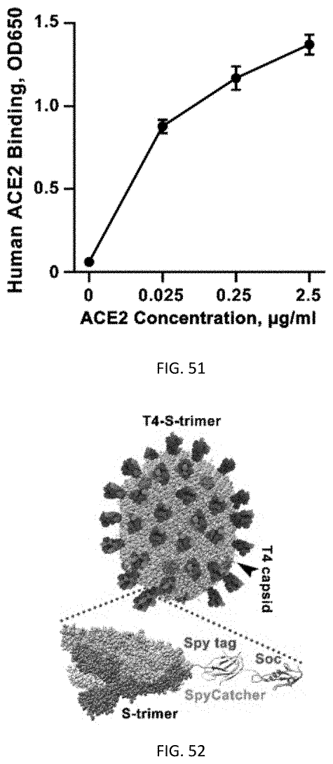

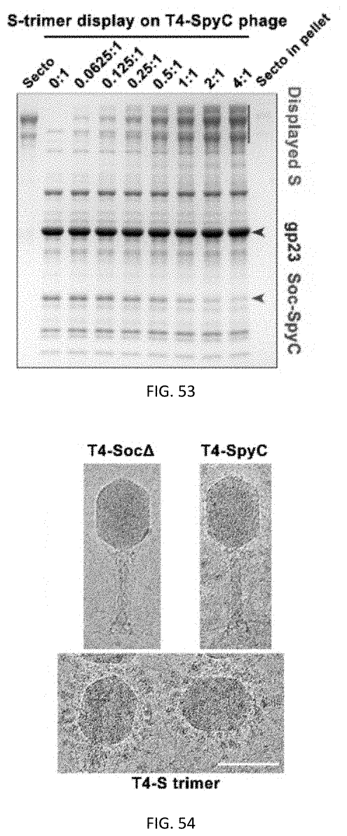

In one embodiment, spike ectodomain (S-ecto) trimers (433.5 kDa) were displayed on T4-Spike-Ee-NP-SocΔ phage. The pre-fusion stabilized hexa-Pro S-ectodomain construct described as above was fused to a 16-aa spytag at the C-terminus and expressed in ExpiCHO cells. The ectodomain trimers secreted into the culture medium were purified by HisTrap affinity chromatography and size-exclusion chromatography. The successful construction of the trimers were confirmed by Size-exclusion chromatography (SEC) as shown in FIG. 49 and Reducing SDS-PAGE (top panel of FIG. 50) and BLUE NATIVE-PAGE (bottom panel of FIG. 50). In FIG. 49, HisTrap affinity purified S-ecto-spy protein from 250 ml of the transfected ExpiCHO cells was loaded on Superdex 200 pg SEC column. S-trimer yield is ˜50 mg per 1 L culture. In FIG. 50, the molecular weight standards (M) in kDa are shown on the left of the gels. IMAC (Immobilized Metal Affinity Chromatography, His) fraction is the material from affinity purification of culture supernatant on a HisTrap column, which was then loaded on the SEC column. These trimers appeared authentic and native-like because: i) the trimers migrated predominantly as a single species and showed no nonspecific aggregation, which usually appears as a separate peak near void volume and as a smeary high molecular weight species on native gel in FIG. 50, and ii) the trimers bound efficiently to human ACE2 receptor, as shown in FIG. 51 and to conformational RBD-specific mAbs, as shown in FIG. 48. Importantly, they were efficiently captured by the spycatcher phage produced as above. The decoration of phage T4 nanoparticles with spike ectodomain trimers with spycatcher tag is schematically illustrated in FIG. 52. Binding was so strong that efficient assembly occurred by simple mixing of trimers and phage even at an equimolar ratio of S-ecto to T4-spycatcher molecules. FIG. 53 shows in vitro assembly of S trimers on T4-SpyCatcher phage at increasing ratios of S-trimer molecules to Soc binding sites ranging from 0:1 to 4:1. Phage and S-trimer were incubated at 4° C. for 1 hr, followed by centrifugation to remove the unbound material. After two washes, the pellet was re-suspended in buffer and SDS-PAGE was performed. The copy number was about 100 S-ecto trimers per phage capsid, according to FIG. 53. In FIG. 53, the displayed Secto trimer was quantified using standard Secto protein, which is not shown in FIG. 53. As shown in FIG. 54, cryo-EM analysis also showed decoration of T4 phage capsids with S-ecto trimers, mimicking the trimers exposed on SARS-CoV-2 virion. In FIG. 54, the scalebar is 100 nm.

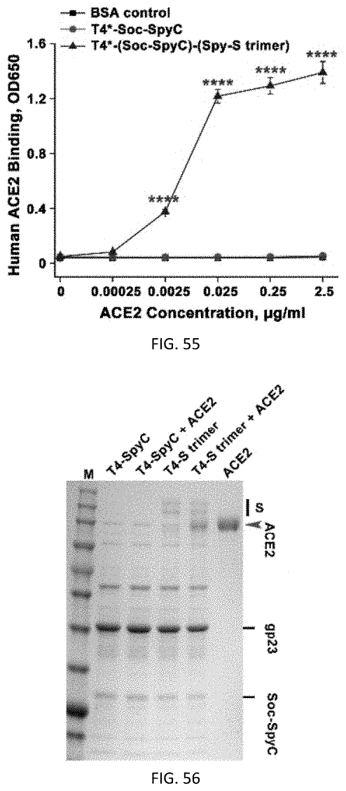

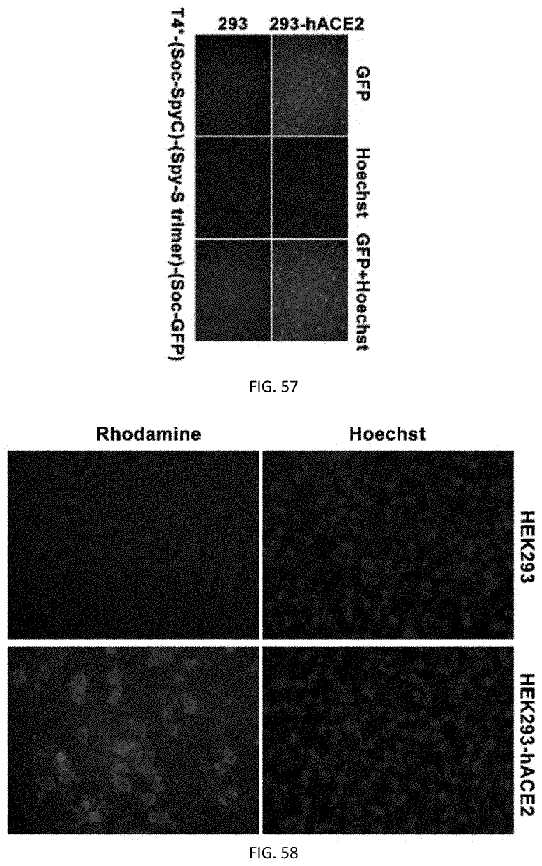

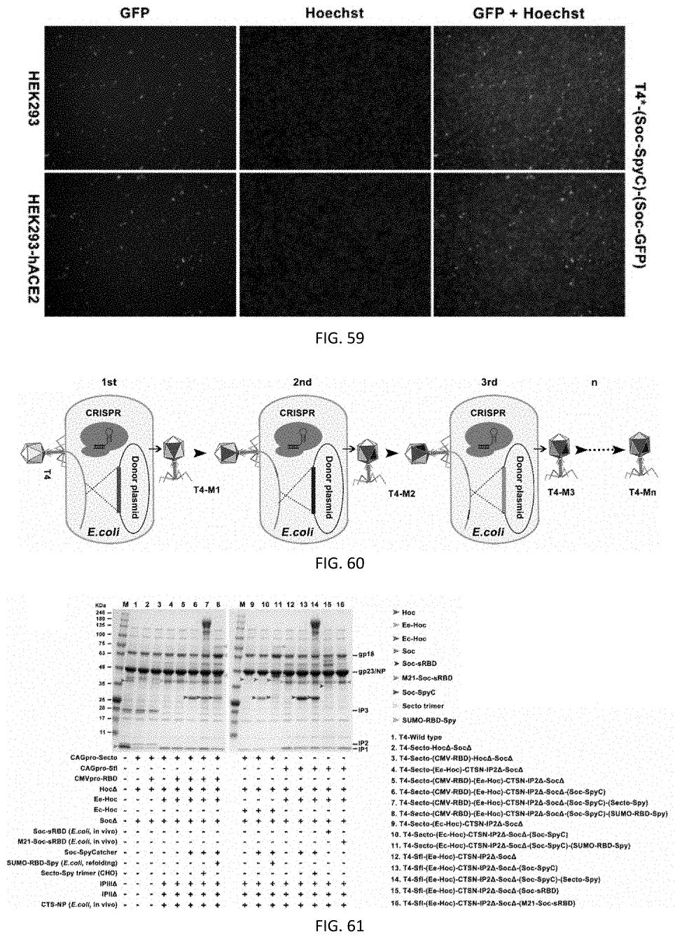

In one embodiment, the trimers-decorated T4 phage efficiently bound to human ACE2 receptor. FIG. 55 shows T4-S-trimer phage binding to ACE2 at various ACE2 concentrations. In FIG. 55, **** refers to P<0.0001. FIG. 56 shows the results of co-sedimentation assay confirming the capture of ACE2 by T4-decorated S trimers. In the co-sedimentation assay, T4-S-trimer particles and ACE2 were incubated at equimolar ratio for 1 hr at 4° C., followed by high speed centrifugation. After two washes, the pellet was re-suspended in buffer and SDS-PAGE was performed. Presence of ACE2 in the pellet was found with these phage particles but not with the control phage lacking S-trimers. In another embodiment, the trimers were co-displayed with GFP, they bind to the ACE2-expressing HEK293 cells. In FIG. 57, the presence of GFP in ACE2-expressing HEK293 cells, which is absent in HEK293 cells without ACE2, indicate the bining to ACE2 by the S-trimer decorated phages. The nucleus was stained with Hoechst. T4* in FIG. 57 indicates T4-(S-ecto)-RBD-NP-Ee-SocA, in which S-ecto and RBD indicated the insertions of gene expression cassettes. The expression of ACE2 on 293 cells but not the control 293 cells was also confirmed by florescent staining, as shown in FIG. 58. To visualize the ACE2 expression, two days after ACE2 plasmid transfection, 293 cells were incubated with RBD, followed by anti-RBD antibody and Rhodamine-conjugated second antibody. To confirm the binding is specific between S trimer and ACE2, binding of T4-GFP control phage without S trimer to ACE2-293 cells was also determined and shown in FIG. 59. As shown in FIG. 59, lack of binding of T4-GFP control phage (without S trimer) to ACE2-293 cells indicates the binding is specific between S trimer and ACE2. No difference in fluorescence was observed between 293 cells with and without ACE2 expression. The nuclei were stained with Hoechst. In FIG. 59, T4* indicates T4-(S-ecto)-RBD-NP-Ee-SocA.

### Immunogenicity and Protective Efficacy of T4-SARS-CoV-2 Vaccine Candidates

In one embodiment, the T4-CoV-2 vaccine candidates generated as above by sequential engineering. FIG. 60 illustrates the steps of sequential engineering. Briefly, wild-type phage was used as a starting phage to infect the bacteria containing designed spacer 1 and donor 1. The resultant T4-mutant 1 (T4-M1) infected the bacteria containing spacer 2 and donor 2 to produce recombinant T4-mutant 2 (T4-M2), which has two insertion/deletion mutations, and so forth. By sequential CRISPR engineering, the phage with multiple desired mutations was created. Each color on phage capsid here represents a mutation. FIG. 61 shows an example of phage sequential CRISPR engineering for creating “universal” SARS-CoV-2 vaccine, with the presence of mutations confirmed by PCR/sequencing and SDS-PAGE. Briefly, a numerous elements, including CAGpromoter-S-ecto insertion, CAGpromoter-S-fl insertion, CMVpromoter-RBD insertion, Hoc deletion, Ee-Hoc insertion, Ec-Hoc insertion, Soc deletion, Soc-sRBD display, M21-Soc-sRBD display, Soc-SpyCatcher display, refolding SUMO- RBD-Spy display, S-ecto-Spy trimer display, IPIII deletion, IPII deletion, and NP encapsidation, were permutated and combined as needed. The resultant SARS-CoV-2 vaccine candidates were characterized by PCR/sequencing and SDS-PAGE, and then can be tested in one animal study. M21 in FIG. 61 indicates a potential T cell 21 aa epitope (SYFIASFRLFARTRSMWSFNP) from SARS-CoV-2 membrane protein.

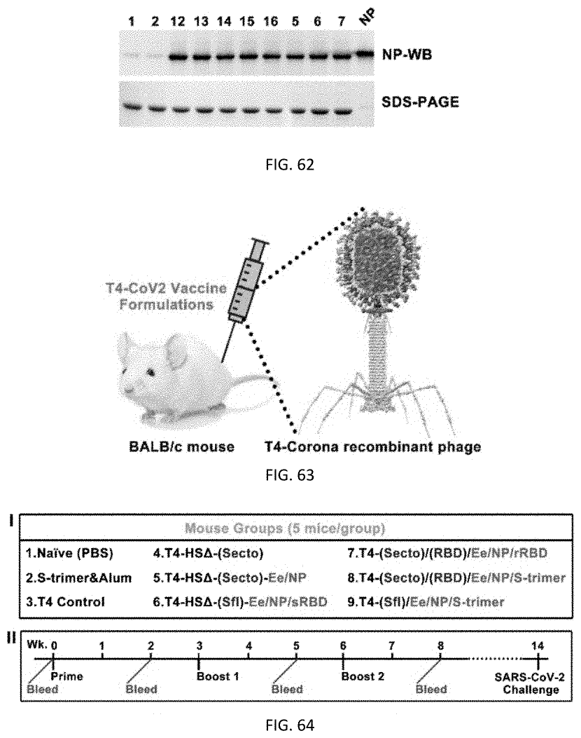

FIG. 62 shows Western Blotting results confirming NP protein encapsidation in the phages containing CTSam-NP insertion at IPIII deletion site. The encapidation of NP cannot be shown on SDS-PAGE as shownin FIG. 61, because of its molecular weight is too close to that of T4 capsid protein gp23. Therefore, Western Blotting was comducted with NP separately and shown in FIG. 62 to confirm the presence of NP protein in NP gene containg phages. The sample numbers in FIG. 62 are as below:

1. T4-Wild type

### 2. T4-Secto-HocA-SocA

### 5. T4-Secto-(CMV-RBD)-(Ee-Hoc)-CTSN-IP2A-SocA

### 6. T4-Secto-(CMV-RBD)-(Ee-Hoc)-CTSN-IP2A-SocΔ-(Soc-SpyC)

### 7. T4-Secto-(CMV-RBD)-(Ee-Hoc)-CTSN-IP2A-SocΔ-(Soc-SpyC)-(Secto-Spy)

### 12. T4-Sfl-(Ee-Hoc)-CTSN-IP2A-SocA

### 13. T4-Sfl-(Ee-Hoc)-CTSN-IP2A-SocΔ-(Soc-SpyC)

### 14. T4-Sfl-(Ee-Hoc)-CTSN-IP2A-SocΔ-(Soc-SpyC)-(Secto-Spy)

### 15. T4-Sfl-(Ee-Hoc)-CTSN-IP2A-SocΔ-(Soc-sRBD)

### 16. T4-Sfl-(Ee-Hoc)-CTSN-IP2A-SocΔ-(M21-Soc-sRBD).

In one embodiment, the vaccine candidates obtained using the sequential engineering method above can be screened for their immunogenicity and protective efficacy in a mouse model. Schematic diagram in FIG. 63 shows Balb/c mice immunized by intramuscular (i.m.) route using T4-SARS-CoV-2 vaccine formulations. FIG. 64 shows the formulations, groups and prime-boost immunization scheme used for mice vaccinations. Panel I of FIG. 64 shows formulations and groups used for mice vaccinations. HSA indicates Hoc deletion and Soc deletion. S-ecto, S-fl, and RBD were inserted mammalian gene expression cassette into T4 genome as DNA vaccine. Ee, S-trimer, or E. coli-produced RBD protein were capsid-displayed, and the NP protein was capsid-encapsidated. Naïve mice and mice immunized with hoc.del-soc.del phage lacking any CoV-2 genes served as negative controls whereas mice immunized with spike trimers adjuvanted with Alhydrogel served as a positive control. Panel II of FIG. 64 shows prime-boost immunization scheme. Balb/c mice (5 per group) were boosted on days 21 and 42 and challenged (intranasal, i.n.) with a mouse-adapted SARS-CoV-2 strain (SARS-CoV-2 MA10)¹² on day 91. The groups are summarized as below:

| | | Gene | |
|---|---|---|---|
| | | expression | Capsid |
| Group No. | Formulation | cassette | display |
| Goup 1 | Naïve (PBS) | | |
| (G1) | | | |
| Goup 2 | S-trimer & Alum | | |
| (G2) | | | |
| Goup 3 | T4 Control | | |
| (G3) | | | |
| Goup 4 | T4-HSΔ-(Secto) | Secto | |
| (G4) | | | |
| Goup 5 | T4-HSΔ-(Secto)- | Secto | Ee and NP |
| (G5) | Ee/NP | | |
| Goup 6 | T4-HSΔ-(SfI)-Ee/ | SfI | Ee, NP and |
| (G6) | NP/sRBD | | sRBD |
| Goup 7 | T4-(Secto)/(RBD)/ | Secto and | Ee, NP and |
| (G7) | Ee/NP/rRBD | RBD | rRBD |
| Goup 8 | T4-(Secto)/(RBD)/ | Secto and | Ee, NP and |
| (G8) | Ee/NP/S-trimer | RBD | S-trimer |
| Goup 9 | T4-(Sfl)/Ee/NP/S- | Sfl | Ee, NP and |
| (G9) | trimer | | S-trimer |

In one embodiment, the vaccine candidates produced using the above method can induce immune responses, as evidenced by the SARS-CoV-2-specific antibody titers. The SARS-CoV-2-specific antibody titers were determined by ELISA using purified proteins, including S-ecto, RBD, NP, or E as coating antigens.

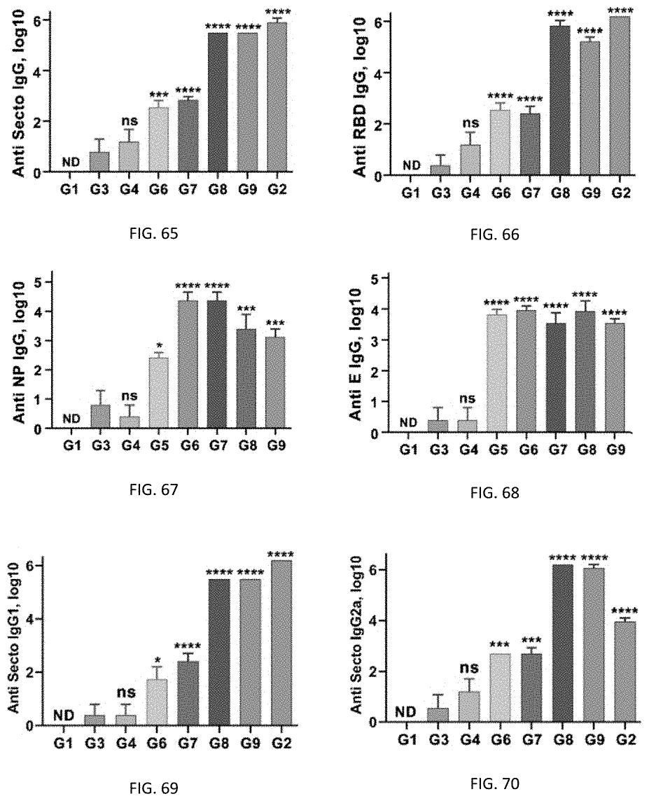

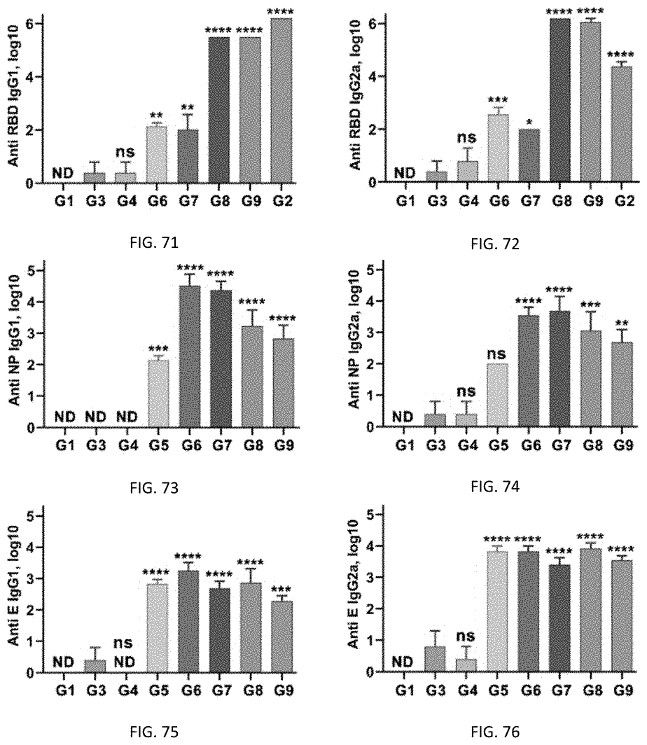

In one embodiment, recombinant phages that delivered CoV-2 DNA alone did not induce significant titers of spike-specific antibodies. FIGS. 65-76 shows the IgG, IgG1 and IgG2a antibody titers, in which the column G4, corresponding to group 4, did not show significant increase in antibody titers. In FIGS. 65-76, * refers to P<0.05, ** refers to P<0.01, *** refers to P<0.001, and **** refers to P<0.0001, compared with phage control group G3. Also in ns (no significance) refers to P>0.05 and ND refers to not detected based on endpoint calculation.

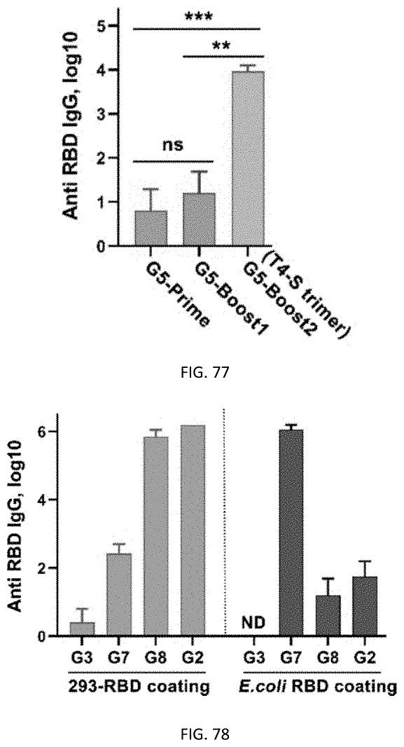

In one embodiment, recombinant phages that delivered CoV-2 DNA did not elicit anti-RBD antibody titers after boost-1 with same particles (CoV-2 DNA). But they were able to elicit significant antibody titers if boost 2 phage nanoparticles were displayed with ectodomain trimers. FIG. 77 shows the anti-RBD IgG titer of group 5 after the boost-1 and boost-2 with T4-S trimer. According to FIG. 77, there is a significant increase in antibody titer after boost-2. In FIG. 77, ** refers to P<0.01 and *** refers to P<0.001.

In one embodiment, strong antibody titers were elicited against T4 nanoparticle-delivered protein or peptide epitopes, either displayed on surface or packaged inside. These include E-specific antibodies, NP-specific antibodies, RBD-specific antibodies, and spike-specific antibodies, as shown in FIGS. 65-68. However, the highest titers, up to an endpoint titer of ˜1.5 ×10⁶, were obtained with phage nanoparticles decorated with the S-ectodomain trimers. Most of these antibodies seem to be specific to RBD since no significant difference was observed between the endpoint titers obtained by using either RBD or S-ecto trimers as the coating antigen, because the antibody titers of the same group were about the same shown in FIGS. 65-66. This result is consistent with the recent studies indicating that immunodominant RBD comprises multiple distinct antigenic sites and is the target of most neutralizing activity in COVID-19 convalescent sera^{8, 9}.

In one embodiment, antibodies elicited were specific to the conformation of the displayed protein. For instance, the antibodies elicited against sRBD or rRBD displayed on phage reacted poorly with the mammalian expressed S-ecto trimer or RBD, as evidenced by the lower antibody titers of groups 6 and 7 (G6 and G7) in FIGS. 65-66. This result is consistent with the antigenicity data described above that the sRBD and rRBD reacted poorly with ACE2 and native conformation-specific RBD mAbs, as shown in FIGS. 47-48. Similarly, the antibodies elicited against S trimers displayed on T4 reacted poorly with the E. coli-produced RBD, as shown in FIG. 78. In FIG. 78, the end point titer of anti-RBD IgG was about 10² using E. coli RBD as the coating antigen, while the end point titer of anti-RBD IgG was about 10⁵ using mammalian RBD as the coating antigen.

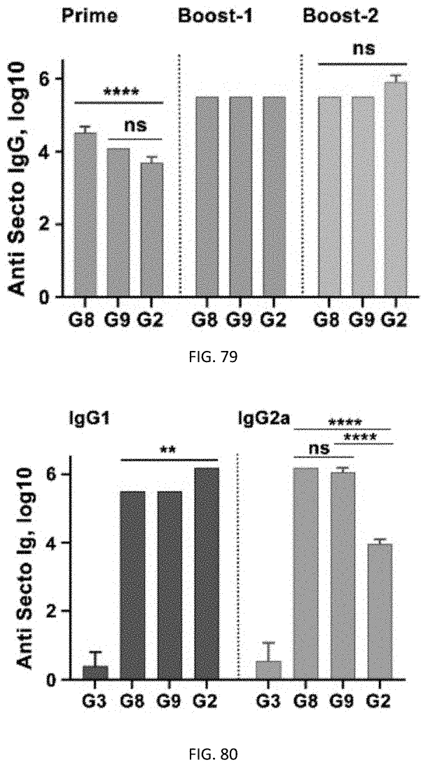

In one embodiment, the endpoint titers elicited by phage-delivered trimers without any adjuvant were as high as those generated using Alhydrogel as an adjuvant. FIG. 79 shows measurement of anti-S-ecto IgG antibody endpoint titers in sera from S-trimer & Alhydrogel (G2) group and T4-S-trimer (G8 and G9) groups at 2 weeks (prime), 5 weeks (boost-1), and 8 weeks (boost-2). In FIG. 79, *** refers to P<0.001.

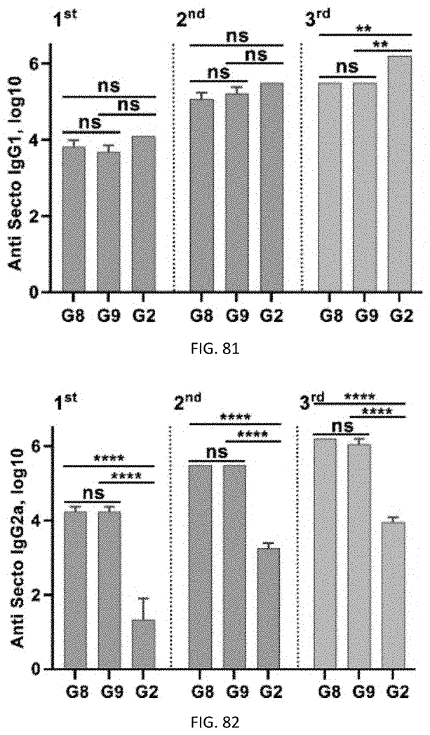

In one embodiment, IgG subclass specificity data indicated that phage nanoparticles stimulated both humoral (TH2) and cellular (TH1) arms of the immune system. In mice, IgG2a subclass represents TH1 response whereas IgG1 class reflects TH2 response. The adjuvant-free T4 nanoparticles (G8 and G9) elicited high levels of both IgG1 and IgG2a classes against all three SARS-CoV-2 antigens, including spike/RBD, E, and NP, as shown in FIGS. 69-76, whereas the Alhydrogel-adjuvanted trimers (G2) predominantly elicited TH2-derived IgG1 class antibodies (anti-spike or anti-RBD). The anti-S-ecto IgG1 and IgG2a subtype antibody titers in sera from S-trimer & Alhydrogel (G2) group and T4-S-trimer (G8 and G9) groups at 8 weeks (boost-2) were compared and also shown in FIG. 80. Similar conclusion can be obtained from FIGS. 81-82, which shows the anti-S-ecto IgG1 (I) and IgG2a (J) antibody titers in the sera from S-trimer & Alhydrogel (G2) group and T4-S-trimer (G8 and G9) groups at 2 weeks (prime), 5 weeks (boost-1), and 8 weeks (boost-2). In FIGS. 80-82, ** refers to P<0.01 and **** refers to P<0.0001.

In one embodiment, phage nanoparticles gave slightly higher TH1-derived IgG2a antibodies than the TH2 derived IgG1 class antibodies, while the alum-adjuvanted mice elicited two orders of magnitude lower IgG2a antibodies, as showin in FIGS. 80-82. While adjuvants that are TH2-biased may lead to lung injury, the TH1-type adjuvants are proposed to alleviate the potential lung immunopathologyl^{10,11}. T4 nanoparticle vaccine with a balanced TH1 and TH2 responses might be optimal for safety and virus clearance, and this point requires further investigation.

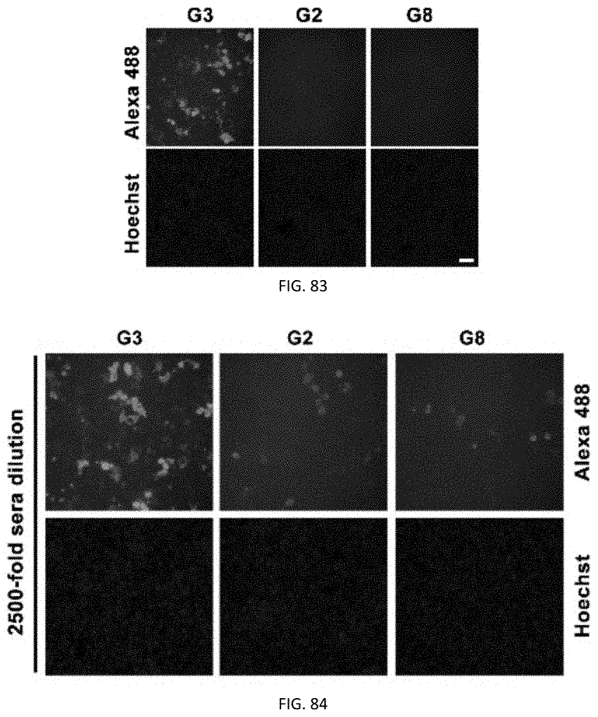

In one embodiment, the T4-stimulated spike-specific antibodies blocked binding of RBD to human ACE-2 expressed on HEK293 cells in a dose-dependent manner. FIGS. 83 and 84 show blocking of native RBD protein binding to 293-ACE2 by sera from phage control group (G3), S-trimer & Alhydrogel group (G2), and T4-S-trimer group (G8). The corresponding sera were diluted 500 fold in FIGS. 83 and 2500 fold in FIG. 84. The RBD binding to 293 surface ACE2 was detected by Alexa® 488 conjugated secondary antibody, while the primary antibody was anti-RBD human IgG.

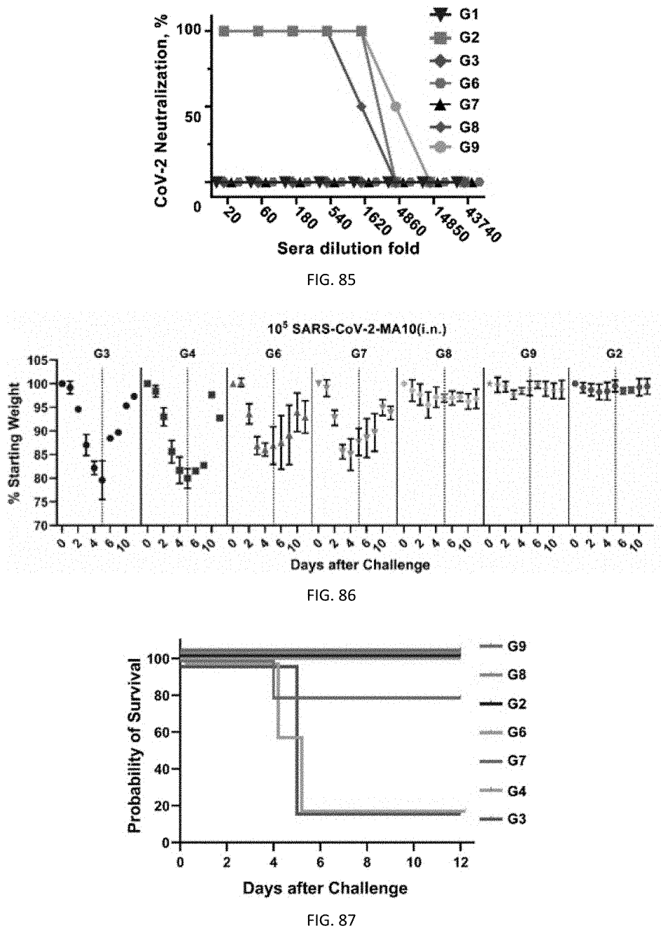

In one embodiment, these antibodies elicited by the engineered phage vaccine candidates described above also exhibited strong virus neutralizing activity as determined by Vero E6 cell cytopathic assay using the live SARS-CoV-2 US-WA-1/2020 strain (BSL-3). FIG. 85 shows neutralization antibody measurement. According to FIG. 85, group 8 and 9 (G8 and G9) can effectively neutralize SARS-CoV-2 at the fold of dilution upto 4860. Infection of Vero E6 cells by SARS-CoV-2 live virus was determined in the presence of mouse sera at a series of threefold dilutions starting from 1:20 (Methods).

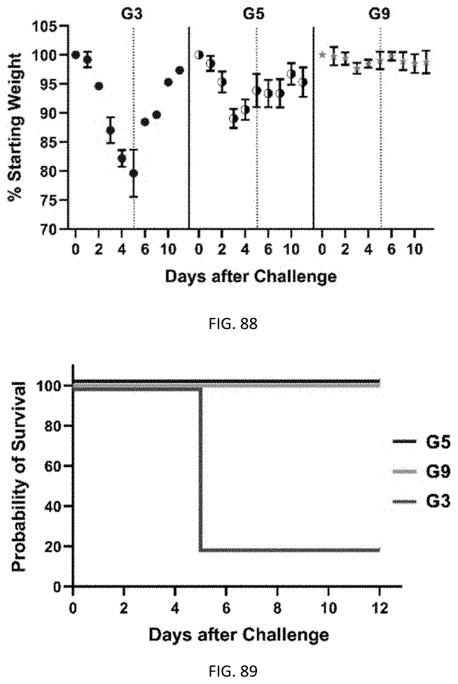

In one embodiment, the neutralization titers correlated with protective efficacy when mice were challenged with mouse-adapted SARS-CoV- 2 MA10 virus12. The protective efficacy can be reflected by the body weight changes of immunized animal and the rate of survival. FIG. 86 shows percentage starting body weight of immunized mice at days post infection with 105 PFU SARS-CoV-2 MA10 through intranasal inoculation (i.n.). In FIG. 86, the dotted line represents percentage starting weight at day 5 post infection, in which mice showed maximum weight loss in control groups. In groups G3 and G4, only 20% mice survived after day 5. Thus, data presented after day 5 are biased toward minor survivors. The data were presented as means ±SD in FIG. 86. According to FIGS. 86, G8 and G9 that have high neutralization titers showed little weight loss, similar to the control G2 group. FIG. 87 shows the survival of mice against SARS-CoV-2 MA10 challenge, in with G8 and G9 with high neutralization titers also showed highest survival. Moreover, the naive and T4 control mice showed a rapid decline in weight loss, up to 25% of their starting weight in five days due to acute viral infection, and then showed mortality or began to re-gain the weight and recover from the infection during the next several days. This rapid weight loss resulted in ˜80% mortality rate, as shown in FIGS. 86 and 87. None of the groups receiving the spike DNA vaccine alone and/or CoV-2 antigens other than spike trimers such as E, NP, or E. coli-expressed RBDs showed significant protection. However, E. coli RBDs combined with E and NP phage groups (G6 and G7) showed less weight loss and higher survival rate than the susceptible control groups (G3 and G4). On the other hand, mice vaccinated with T4-decorated trimers showed full protection from acute infection. None showed mortality or the rapid rate of weight loss that is characteristic of acute infection. The weight loss in these mice was quite small, for both these groups, as well as the positive control group vaccinated with Alhydrogel-adjuvanted trimers. Additionally, the mice boosted with just one dose of T4-trimers also showed partial protection, as shown in FIGS. 88-89. The weight loss was in between the unprotected and protected groups, clearly correlating protection with native spike-specific antibodies.

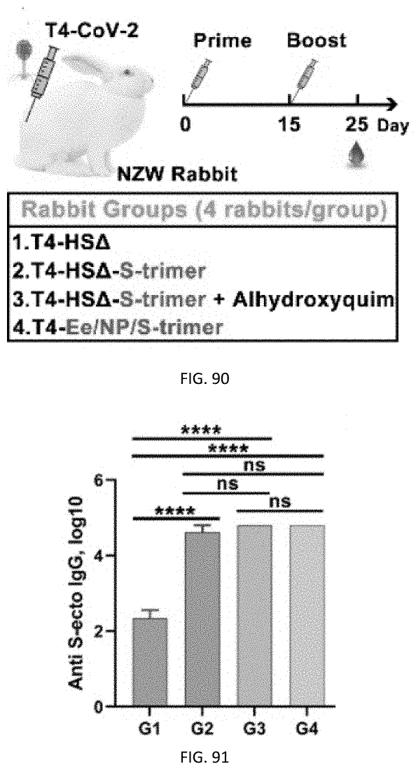

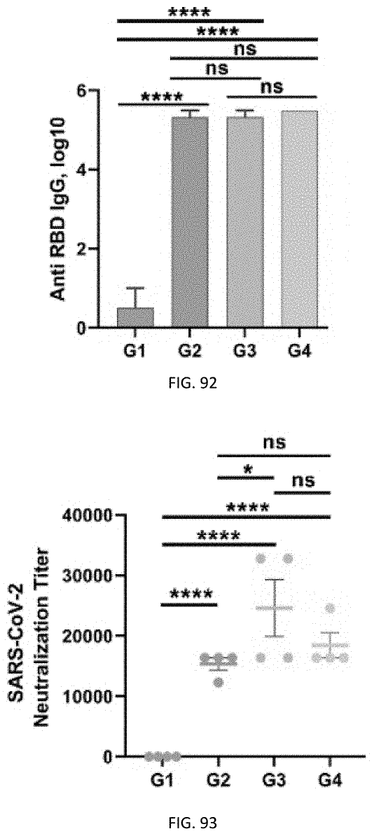

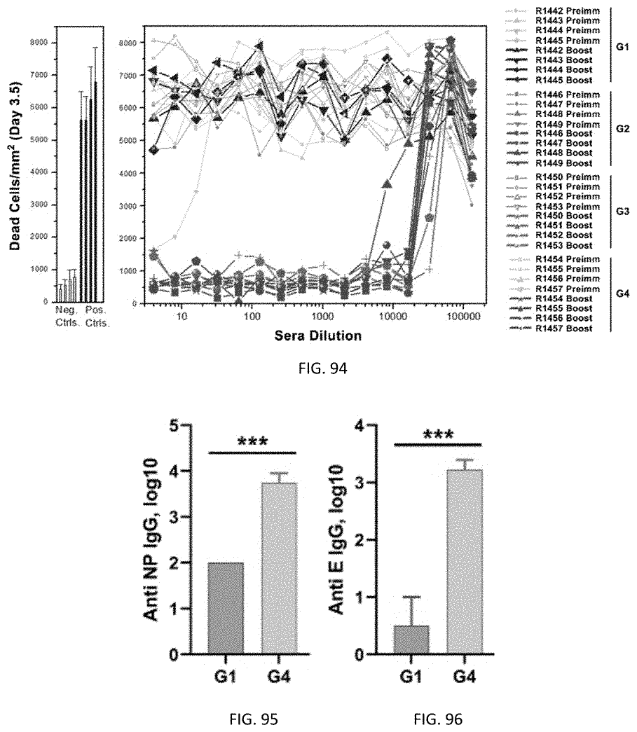

In one embodiment, the most effective vaccine candidate down-selected from the mouse study, the T4 phage-decorated trimers, was evaluated for its ability to induce virus neutralizing titers in a different animal model, including the New Zealand White rabbit. The immunization of rabbits followed the formulations, groups, and prime-boost immunization scheme shown in FIG. 90. In FIG. 90, HSA indicates Hoc deletion and Soc deletion. Red color indicates the capsid-displayed Ee, S-trimer, or the capsid-encapsidated NP protein. Rabbit immunized with hoc.del-soc.del phage served as negative control. This candidate, again without any adjuvant, generated robust spike/RBD-specific antibodies and virus neutralization titers in rabbits, ˜4-6 greater than those obtained in mice, as shown in FIGS. 91-94. In FIGS. 91 and 92, **** refers to P<0.0001 and “ns” (no significance) refers to P>0.05. In FIG. 93, serial dilutions of serum from immunized rabbit were assessed for neutralization of live SARS-CoV-2 (isolate USA-WA1/2020). The neutralization titers were calculated as the reciprocal dilution where infection (cytopathic effect) was reduced by more than 95% relative to infection in the absence of serum. FIG. 94 shows comparison of NZW rabbit neutralization antibody titers in pre-immune and boost sera (10 days after boost). Infection of Vero E6 cells by Live SARS-CoV-2 (isolate USA- WA1/2020) was determined in the presence of rabbit sera at a series of twofold dilutions starting from 1:4 (Methods). Culture medium only and CoV-2 virus only was used as negative control and positive control, respectively. R1442 to R1457 represents the tag number of each rabbit. The data in control groups were presented as means±SD of 32 wells. The data in rabbit sera groups were showed as means of duplicates. In one embodiment, inclusion of displayed Ee peptide and packaged NP into the nanoparticle elicited broad immune responses against both these antigens, as shown in FIGS. 95-96. In FIGS. 95-96, *** refers to P<0.001.Addition of TH1-biased adjuvant Alhydroxyquim-II (ref) only slightly enhanced the antibody titers, also as shown in FIGS. 91-93.

In one embodiment, the phage vaccine candidate selected using the presently disclosed method was highly effective, which generated robust virus neutralization titers in two different animal models, mouse and rabbit, which also resulted in complete protection against acute viral infection in mice.

In one embodiment, a “universal” vaccine platform was developed centered around the bacteriophage T4 nanoparticle. As demonstrated in the present disclosure, a number of special features would make this a powerful platform to rapidly generate vaccine candidates against any emerging and pandemic pathogen in future.

In one embodiment, a series of recombinant phages containing SARS-CoV-2 gene insertions were generated in mere days by CRISPR genome engineering using a combination of type II Cas9 and type V Cpf1 nucleases. This combination provides built-in choices for spacers as well as for efficient cleavage of T4 genome that is extensively modified by cytosine hydroxymethylation and glycosylation, for attaining near 100% success^{13, 14}.

In another embodiment, a large amount of genetic and structural space available in phage T4 was exploited to incorporate CoV-2 DNAs, peptides, proteins, and complexes into the same nanoparticle. In a preferred embodiment, at least 6 kb full-length spike gene expression cassette, 2.7 kb RBD gene expression cassette, and about 1.3 kb nucleocapsid gene were inserted into the same genome by replacing certain nonessential genetic material. In one embodiment, the genetic space was further expanded by inserting Hoc and Soc fusions, and/or replacing additional nonessential segments that span across the genome. In another embodiment, up to 155 copies of a 12-aa Ee peptide and ˜100 copies of 433.5 kDa S-trimers were displayed on the same nanoparticle while ˜70 molecules of 50-kDa NP were packaged inside the structure. These represent an extremely large payload carried by any vaccine delivery vehicle reported thus far.

In one embodiment, different areas of phage nanostructure were utilized for placing different vaccine cargos. In a preferred embodiment, the tips of Hoc fibers with ˜170 Å reach were used to display short 12-aa Ee peptide epitopes as this would allow efficient capture by antigen presenting cells, B cells, and T cells¹⁵. In one embodiment, these vaccine candidates resulted in strong antibody titers. At the same time, the nucleocapsid protein was co-packaged with phage genome as this mimics NP's natural environment as an RNA binding protein.

In one embodiment, the T4 platform could be readily adapted to mammalian-expressed proteins, which might be essential for proper folding and glycosylation, as in the case of spike trimers, through formation of highly efficient spycatcher-spytag bridges. As the Cryo-EM structure showed, such spikes anchored to capsid with their RBDs well-exposed in some respects mimic the spikes present on SARS CoV-2 virion and might provide a more native context for stimulating effective immune responses.

In one embodiment, alternative strategies such as pre-expression from a plasmid and display through subsequent phage infection provided additional advantages such as enhancing copy number and better control over folding. As demonstrated, this approach resulted in 3-6 fold higher copy number of Soc-spycatcher on phage capsid. However, RBD still remained poorly folded.

In one embodiment, sequential engineering generated a pipeline of SARS-CoV-2 vaccine candidates in mere weeks, and allowed down-selection of the best vaccine candidate, phage decorated with trimers, in a single animal experiment. However, the DNA-alone vaccines failed to elicit immune responses.

In one embodiment, unlike the DNAs, the peptide/protein epitopes, either displayed on surface or packaged inside, generated robust immune responses. The strong virus neutralization titers and ACE-2 blocking titers elicited by T4-delivered trimers in two different animal models, mouse and rabbit, are particularly noteworthy. These responses correlated with protective efficacy where all the vaccinated mice were protected from acute viral infections.

In one embodiment, the T4 phage vaccines generated balanced TH1 and TH2 derived antibody responses against all three CoV-2 antigens tested. In fact, T4 seems to have a slight TH1-bias, a desirable property that distinguishes this platform from other subunit vaccine platforms and adjuvant systems. The TH1-bias might be because the phage-decorated antigens are recognized as PAMPs present on natural viruses thereby triggering host responses through Toll-like receptors (TLR) 2 mediated innate immunity pathways. In one embodiment, TLR2 and TLR4 pathways are stimulated by the engineered phage vaccines in in vitro cultured cells (data not shown).

In one embodiment, the T4 nanoparticle vaccine does not require an adjuvant to stimulated robust anti-CoV-2 immune responses, as demonstrated in two different animal models. Therefore, in addition to reducing cost and manufacturing complexity, the adjuvant-less T4 vaccine formulations are less reactogenic that is often associated with the chemical adjuvants used in traditional vaccine formulations. In numerous immunizations with T4 phage performed in a variety of animal models including mouse, rat, rabbit, and macaque, no significant reactions were noted.

In one embodiment, one of the best T4 vaccine candidates elicits, in addition to spike-specific antibodies, broad antibody responses against two additional virion components, E that is exposed on the surface and NP that is abundantly present on CoV-2 infected host cells. In one embodiment, the spycatcher phage could serve as a backbone to capture ectodomain trimers from other coronaviruses, or any spytagged antigen(s) from other infectious agents. Thus, it is conceivable that different vaccine formulations can be “created” at the site of administration by mixing the spycatcher T4 phage with the desired antigen(s) combinations. Co-carrying multiple and distinct antigens on the same nanoparticle can increase the breadth of the elicited immune responses16,17. Since the S-ecto, Ee and NP are conserved in other coronaviruses such as SARS-CoV-1, the T4 vaccine platform might be considered for expanded protection.

In one embodiment, a versatile “universal” vaccine design template by which vaccine candidates for any emerging or pandemic infectious agent can be rapidly generated, was developed and evaluated in animal models. As T4 is a highly stable nanoparticle, has good safety profile and no pre-existing antibodies in humans, manufactured for mass production at a relatively low cost, it provides a robust platform to rapidly generate effective vaccines against epidemic- and pandemic-causing pathogens in the future, particularly when multivalent vaccine candidates are essential to protecting global communities.

Having described the many embodiments of the present disclosure in detail, it will be apparent that modifications and variations are possible without departing from the scope of the invention defined in the appended claims. Furthermore, it should be appreciated that all examples in the present disclosure, while illustrating many embodiments of the invention, are provided as non-limiting examples and are, therefore, not to be taken as limiting the various aspects so illustrated.

### EXAMPLES

### Example 1

### DNA, Bacteria, and Bacteriophage

The expression vector pET28b (Novagen®, MA) was used for donor plasmid construction and protein expression plasmid construction. LbCpf1 and SpCas9 plasmids were constructed for spacer cloning^{13, 14}. Briefly, SpCas9 plasmids were constructed by cloning spacer sequences into the streptomycin-resistant plasmid DS-SPCas (Addgene® no. 48645). Sequences of the spacers are shown in table below. LbCpf1 plasmid was constructed by replacing Cas9 and its spacer cassette in SpCas9 plasmid with Cpf1 and spacer cassete.

| Spacers | Sequence (5′-3′) | GC, % | |
|---|---|---|---|
| Cpf1-39-56-sp1 | gttgcattaatcagcatcag | 40 | 39-56 11 Kbp deletion |
| Cpf1-39-56-sp2 | cgcccttgaagttccttctg | 55 | |
| Cpf1-FarP7K-sp1 | tccactccaagatgctccat | 50 | FarP 7 Kbp deletion; |
| Cpf1-FarP7K-sp2 | aaaccgttcaagagtttttg | 35 | CAG-CD5-Sf1 or |
| Cpf1-FarP7K-sp3 | aatttagcactcgtggagat | 40 | CAG-CD5-Secto |
| Cpf1-FarP7K-sp4 | tcgcccgaatgaatccagtt | 50 | insertion |
| Cpf1-FarP7K-sp5 | ggaagaatccgttaatcgtc | 45 | |
| Cpf1-FarP7K-sp6 | ccagtgagttttcacacgaa | 45 | |
| Cpf1-FarP18K-sp1 | cactgatgaagaaacggtgt | 45 | FarP 18 Kbp deletion; |
| Cpf1-FarP18K-sp2 | tctactgtaatcatgtccca | 40 | |
| Cpf1-FarP18K-sp3 | tcgttggttcattatacacc | 40 | |
| Cpf1-FarP18K-sp4 | gaattaatcgtgctgataca | 35 | |
| Cpf1-SegF-sp1: | ttccttctccaccctgacca | 55 | SegF deletion; |
| Cpf1-SegF-sp2: | atgcagatattagctcacgt | 40 | CMV-RMD insertion |
| Cpf1-SegF-sp3: | accatcgtattttataatta | 20 | |
| Cpf1-Hoc-sp1: | cagttgatataactcctaaa | 30 | Hoc deletion; Ee |
| Cpf1-Hoc-sp2: | atcaataacccctgtaggtg | 45 | or Ec insertion |
| Cpf1-Hoc-sp3: | gttatgtactaaaaggacct | 35 | |
| Cpf1-Hoc-sp4: | gaaactggtatcatctatac | 35 | |
| Cpf1-Soc-sp1: | agcagaaattagatggaaat | 30 | Soc deletion |
| Cpf1-Soc-sp2: | atattaacataaccgcgagt | 35 | |
| Cpf1-Soc-sp3: | cagcaatccattcagtacgt | 45 | |
| Cpf1-Soc-sp4: | tggaaagtaactggttaata | 30 | |
| Cpf1-Mrh2-sp1: | ttcattacatgtcgtgaaat | 30 | SpyCatcher or RBD |
| Cpf1-Mrh2-sp2: | gatattatcatttcacgaca | 30 | insertion |
| Cpf1-Mrh2-sp3: | aattcgacttgcttctcacc | 45 | |
| Cpf1-IPIII-sp1: | aagtcggaagcctttgtagc | 50 | IPIII deletion; |
| Cpf1-IPIII-sp2: | tgcttggcaaattcaagacc | 45 | NP insertion |
| Cpf1-IPIII-sp3: | ctgatcggtaggtccactca | 55 | |
| Cpf1-IPIII-sp4: | ctacagaagcttcggcaata | 45 | |
| Cpf1-IPII-sp1: | cttctaagttcggcatgtct | 45 | IPII deletion |
| Cpf1-IPII-sp2: | ttacggtctttatcgggcaa | 45 | |
| Cas9-IPIII-sp1: | atggaaaggtcttgatgcaa | 40 | IPIII deletion; |
| Cas9-IPIII-sp2: | attatcaatgacccatttac | 30 | NP insertion |
| Cas9-IPIII-sp3: | ggcctttactacagaagctt | 45 | |

The DNA fragment containing NP and RBD were codon-optimized for E.coli expression and synthesized by GeneArt (Thermo Fisher®). The plasmids containing wild-type SARS-CoV-2 Spike (S) gene and S-ecto-6P gene were provided by Dr. Kizzmekia S. Corbett (National Institutes of Health) and Dr. Jason S. McLellan (University of Texas, Austin). The RBD gene for mammalian expression was amplified from the wild-type Spike (S) gene. SpyCatcher/Spy-tag and SUMO containing plasmid were purchased from Addgene® (#133449 and #111560).

E. coli strain DH5a cell (hsdR17 (rK−mK+) sup²) (NEB®) was used for all the clone construction. The E. coli BL21-CodonPlus (DE3)-RIPL (Novagen®, MA) was used for the expression of recombinant proteins. P301 (sup⁰) and B834 (hsdRB hsdMB met thi sup⁰were used for the propagation and recombination of phages without amber mutations. The BL21 (DE3) RIPL transformed with amber-suppressor plasmid and E. coli B40 (sup¹) were used for the propagation and recombination of phages with amber mutations (CTS-amber-NP). Wild-type T4 phage was used as a starting phage of CRISPR engineering and propagated on E. coli P301 or B834.

### Example 2

### Plasmid Construction

CRISPR-LbCpfl/SpCas9 plasmid was constructed based on the streptomycin-resistant plasmid DS-SPCas (Addgene® no. 48645)^{13, 14}. The spacer containing fragments were prepared by annealing and extension of two amplified DNA fragments containing 26 bp complementary nucleotides (overlap extension PCR). The spacer fragment digested by restriction enzymes XhoI&EagI was cloned into linearized LbCpf1/SpCas9 plasmid. Sequences of the spacers are shown in the table above in Example 1.

The donor plasmids for deletion/insertion, including Hoc-del, Soc-del, FarP7K-del, FarP18K- del, 39-56 11K-del, SegF-del, IPIII-del, IPII-del, E insertion (Hoc site), Soc-SpyCatcher/RBD insertion (Soc site), CTS-amber-NP insertion (IPIII site), CAG-S-fl/S-ecto insertion (FarP7K site), and CMV-RBD insertion (SegF site), were constructed using overlap extension PCR. Briefly, the corresponding ˜500 bp homology arms (left and right) were amplified from T4 genome DNA, stitched, and cloned into pET28b linearized with BglII and XhoI to generate the deletion donor plasmid. For the insertion donor plasmid, the insertion fragment, left homology arm, and right homology arm, which contains 25 bp complementary nucleotides each other, were stitched by annealing and extension. The BglII&XhoI digested donor fragment was cloned into pET28b.

The Soc-fused expression plasmids, including Soc-SpyCatcher, Soc-RBD, RBD-Soc, SUMO- Soc-Spy, and Soc-truncated RBD (RBD67, RBD106, RBD135, RBD162, RBD181, and RBD197), were constructed by two rounds of cloning. First, the MCS (NcoI, NdeI, NheI, and BmtI)-linker (4GGS)-Soc-linker (2GGGGS)-MCS (HindIII, EagI, NotI, and (hop was amplified using Soc- plasmid template, and cloned into the pET28b DNA linearized with Ncol and XhoI restriction enzymes to generate pET28b-MCS-L-Soc-L-MCS. Second, SpyCatcher, SUMO, Spy, and/or various RBDs were amplified and inserted to 3′- or 5′- MCS of pET28b-MCS-L-Soc-L-MCS as needed. CTS-NP and Ee-Hoc expression fragments were amplified using synthesized NP and T4 genomic DNA respectively, and cloned into Ncol&XhoI-linearized pET28b.

The plasmids for expression in mammalian cell, including pCMV-CDS-RBD, pCAG-CDS-S-fl, pCAG-CDS-S-ecto-6P, and pCAG-CDS-S-ecto-6P-Spytag, were constructed. The RBD fragment was amplified using the wild-type spike gene and CD5 secretion leading peptide (MPMGSLQPLATLYLLGMLVASVLA) was added to the N terminus of RBD by PCR. The CD5-RBD was cloned into pAAV vector (Cell Biolabs) using HindIII and XhoI to construct pCMV-CD5-RBD. For the construction of pCAG-CD5-S-fl, pCAG-CD5-S-ecto-6P, and pCAG-CD5-S-ecto-6P-Spytag, plasmids pCAG-S-fl and pCAG-S-ecto-6P were used as template and backbone. The CD5 fragment was cloned into N terminus of S-fl or S-ecto-6P using KpnI and EcoRI. Similarly, Spytag (RGVPHIVMVDAYKRYK) was cloned into the C terminus of S-ecto using BamHI and XhoI. All the constructed plasmids were sequenced to confirm correct fragment insertion (Retrogen®, CA).

### Example 3

### Plaque Assay

Plaque assay was applied to determine the efficiency of the individual spacers to restrict T4 phage infection. The CRISPR-Cpfl/Cas9 spacer plasmid was transformed into E. coli strains B834 or B40. The serial diluted T4 phages, which was in the range of 10¹ to 10₇ in 100 μl PI-Mg buffer (26 mM Na₂HPO₄, 68 mM NaCl, 22 mM KH₂PO₄, 1 mM MgSO₄, pH 7.5), was mixed with 350 μl of spacer-containing E. coli (10⁸ cells/ml). E. coli cells without spacer were used as control. After incubation at 37° C. for 7 min, 3.5 ml of 0.75% top agar with spectinomycin (50 μ/mL) was added into each tube, mixed, and poured onto LB plate. The plates were incubated at 37° C. overnight to produce plaques. The plaque-forming units (pfu) were counted on each plate and the efficiency of plating (EOP) was determined by dividing the pfu produced from infection of E. coli containing a spacer by the input pfu.

### Example 4

### CRISPR-Mediated T4 Gene Editing

The CRISPR-Cpfl/Cas9 spacer plasmid and the corresponding donor plasmid were co-transformed into E. coli strains, either B834/P301 without amber suppressor or B40/RIPL with amber suppressor as needed. Single-plasmid transformed E. coli cells, either with the donor plasmid or with the CRISPR spacer plasmid, were used as controls. An appropriate amount of T4 phages, which were determined by the EOP as described above, were added to E. coli cells containing spacer&donor and incubated for 7 min at 37° C. After adding 3.5 ml 0.75% top agar with 50 μg/ml spectinomycin and 50 μg/ml kanamycin, the infection mixture was poured onto LB plate and incubated overnight. Single plaque, which was named G1 (Generation 1) was picked using a sterile Pasteur glass pipet and transferred into a 1.5 ml Eppendorf tube containing 200 μl of PI-Mg buffer. After 20 min incubation at room temperature with gently vortexing every 5 mins, serial diluted G1 phages were used to infect spacer-containing E. coli cells (50 μg/ml spectinomycin). The resultant single G2 plaque was picked and used to infect E. coli cells (without spacer or donor) to produce G3 phages. Single G3 plaque was picked into 200 μl of PI-Mg buffer. PCR analysis was applied to check T4 DNA deletion or foreign gene insertion. One microliter G3 phages were denatured at 94° C. for 8 min and used as a template for PCR using Phusion High-Fidelity PCR Master Mix (Thermo Fisher®). The amplified DNA fragment was purified using QlAquick® Gel Extraction Kit (Qiagen®) and then was sequenced (Retrogene®). The sequencing confirmed G3 phages were added a few drops of chloroform, and stored at 4° C. as zero stocks for the next study. More rounds of CRISPR gene editing can be similarly introduced into the same phage as described above.

### Example 5

### Phage Production

E. coli strains B40 or B834 were used for the production of amber-phage or non-amber-phage, respectively. Fresh overnight-cultured E.coli cells were inoculated in 1 L of Moore's media (20 g tryptone, 15 g yeast extract powder, 2 g dextrose, 8 g NaCl, 2 g Na₂HPO₄, 1 g KH₂PO₄, dissolved in 1 L MQ water) at 1/50 dilution, and then cultured at 37 C for 2-2.5 hrs in a shaker incubator at 200 RPM. When the cells reached the density of ˜4×10⁸/ml, phages were added at a multiplicity of infection (MOI) 0.5. The infection mixture was cultured at 37 C, 200RPM, for another 2.5 -3 hours and periodically checked under the microscope. The shape of phage-infected cells usually is changed from long bacilli to short dumbbell shape.

When the cell number started dropping, the culture was transferred to Sorvall GSA bottle and centrifuged at 27,504 g for 1 hr at 4 C. The supernatant was discarded and the pellet was resuspended in 50 ml PI-Mg buffer containing 10 μg/ml DNase I and one tablet of protease inhibitor cocktail. The resuspended pellet was added with 5 ml chloroform and incubated at 37 C for 1 hr to lyse the bacteria and release the phage. Then the debris was removed by low-speed centrifugation at 4,302 g for 10 min. The phage-containing supernatant was transferred to a sterilized falcon tube and the pellet was discarded. The titer of this phage stock was determined using B40 or B834. The working stock of produced phages can be stored at 4° C. for long periods of time (a few drops of chloroform added), or used as seed phage to make Spy-Catcher or Soc-RBD induction-displayed phage, or purified as vaccine candidate for animal study as described below.

### Example 6

### Phage Purification

The produced ˜50 ml phage stock was distributed into 5 Corex glass tubes (10 ml each) and seal with parafilm. The phage stock was centrifuged at 34,540 g for 1 hr using a Sorvall® SS34 rotor. The supernatant was discarded and the phage-containing pellet was resuspended with 1-3 ml PI-Mg buffer plus 5 ul Benzonase overnight at 4 C. Next, the resuspended phages were loaded on CsC1 gradient solution and centrifuged at 152,000 g for 1 hr in a Beckman® ultracentrifuge using a SW 55 Ti swinging bucket rotor. The purified phage band localizes between CsC1 densities 1.46 and 1.55, and was collected by inserting a syringe right below the phage band and aspirating the band. The phages were dialyzed in high-salt Tris-Mg buffer (10 mM Tris-HCl, pH 7.5, 200 mM NaCl, 5 mM MgCl₂) for 4 hrs followed by low-salt Tris-Mg buffer (10 mM Tris-HCl, pH 7.5, 50 mM NaCl, 5 mM MgC12) overnight. The second-round CsC1 centrifugation and dialysis of purified phages were applied to obtain purer phages. Two-round- CsCl-purified phages were further purified by passing through a 0.22 um filter unit to remove any minor contaminants. The phage concentration and copy numbers of displayed antigens were examined by 4-20% SDS-polyacrylamide gel electrophoresis (SDS-PAGE). The phage genomic DNA was released and digested by treatment with frozen-thaw and Benzonase before SDS-PAGE.

### Example 7

### Production of Soc-SpyCatcher or Soc-RBD In-Vivo Induction-Displayed Phage

E. coli BL21 (DE3) RIPL transformed with T7-Soc-Spycatcher (or Soc-RBD) plasmid was used for Soc-Spycatcher (or Soc-RBD) displayed phage production. E. coli BL21 (DE3) RIPL co-transformed with T7-Soc-Spycatcher (or Soc-RBD) plasmid and amber suppressor plasmid was used for the production of Soc-Spycatcher (or Soc-RBD) displayed and NP protein packaged phage. Briefly, the RIPL cells were inoculated in 1 L of Moore's Media at 1/50 dilution with appropriate antibiotics as needed (RIPL-Soc-Spycatcher: 50 μg/ml Kanamycin+37 μg/ml Chloramphenicol; RIPL-Soc-Spycatcher-Amber Suppressor: 50 μg/ml Kanamycin +37 μg/ml Chloramphenicol +100 μg/ml Ampicillin). The culture was incubated at 37° C., 200 RPM, for 2.5-3 hrs. When the cells reached the density of ˜4×10⁸/ml, 0.5 mM IPTG was added for induction. At 10 min post IPTG addition, the corresponding phages (Hoc-del/Soc-del/IPII-del/IPIII-del) were added to infect cells at MOI 0.5. The culture was further incubated at 37 C, 200 RPM, for 3 hours. The following production and purification procedures are the same as described above.

### Example 8

### Endotoxin Measurement

The LAL chromogenic endotoxin quantitation kit (Thermo Fisher®) was used to measure the amount of endotoxin in phage sample using the Limulus Amebocyte Lysate (LAL) assay according to the manufacturer's instructions. Phage samples were diluted with a 2-fold dilution series beginning with an initial 10¹⁰ particles/50 μl in endotoxin-free water. LAL reagent was added and incubated at 37 C, followed by the addition of chromogenic substrate solution. After 6 mins incubation, stop reagent was added and the absorbance was measured at 405 nm. The endotoxin concentration of phage sample was determined using the formulated standard curve. The endotoxin threshold for phage immunization was <0.5 EU/10¹⁰ particles.

### Example 9

### S Trimer Expression and Purification

Plasmid pCAG-CDS-S-ecto-6P-Spytag was transiently transfected into ExpiCHO cells using ExpiFectamine CHO transfection kit (Thermo Fisher®). After 18-22 hours of transfection cells were supplemented with ExpiCHO Feed and Enhancer and grown at 32° C. according to the manufacturer's High Titer protocol. Cultures were harvested 8-10 days after transfection by centrifuging the cells at 3000 g for 20 minutes at 4 C. The supernatant was clarified through a 0.22 μm filter and then loaded on a HisTrap HP column (Cytiva®) previously equilibrated with wash buffer (50 mM Tris-HC1, pH 8.0, containing 300 mM NaCl and 20 mM imidazole), at a flow rate of 1 ml/minute, using AKTA® Prime-Plus liquid chromatography system (GE® Healthcare). Protein-bound column was washed with wash buffer until the UV absorbance reached the baseline to remove non-specifically bound proteins. The trimers were eluted using a 20 mM-300 mM linear gradient of imidazole. HisTRAP eluted peak fractions were pooled and applied to a Hi-Load 16/600 Superdex-200 (preparation grade) size exclusion column (GE® Healthcare) equilibrated with the gel filtration buffer (50 mM Tris-HCl, pH 8, 150 mM NaCl) to obtain further purified trimers, using the AKTA® FPLC system (GE® Healthcare). Eluted fractions were collected, filtrated by 0.22 um filter unit, flash-frozen, and stored at ˜80° C. until use for the Soc-spycatcher mediated display on various phage vaccine candidates.

### Example 10

S Trimer or rRBD Display on T4-SpyCatcher Phage

In vitro display of S-trimer/rRBD on the T4-SpyCatcher phage was assessed by the co-sedimentation^{18, 19}. Briefly, two-round CsC1 purified and 0.22 um filtered phage particles were sedimented for 45 min at 34,000 g in Protein-LoBind Eppendorf tubes, washed twice with sterilized phosphate-buffered saline (PBS) buffer (pH 7.4), and resuspended in PBS buffer (pH 7.4). S-trimer/rRBD was sedimented for 25 min at 34,000 g to remove possible aggregates. T4-SpyCatcher phages were incubated with S-trimer/rRBD proteins at 4° C. for 1 hr. The mixtures were sedimented by centrifugation at 34,000 g for 45 min, and unbound proteins in the supernatants were removed. After washing twice with excess PBS to further remove the unbound protein and any other minor contaminants, the phage pellets containing the displayed proteins were incubated at 4° C. overnight and then resuspended in PBS. For rabbit animal studies, fifty microliters of phage-trimer particles were added to blood agar (TSA with sheep blood) to examine any contamination of a wide variety of fastidious microorganisms. LPS level was also examined using LAL Chromogenic Endotoxin Quantitation Kit (Thermo Fisher®). The resuspended pellets were analyzed using Novex 4-20% SDS-SDS-PAGE mini gel (Thermo Fisher Scientific®, Waltham, Mass.) to quantify the S trimer/rRBD copies. After Coomassie Blue R-250 (Bio-Rad®, CA) staining and destaining, the protein bands on SDS-PAGE gels were scanned and quantified by ChemiDoc™ MP imaging system (BioRad®) and image J. The copy numbers of SpyCatcher and displayed S-trimer/rRBD molecules per capsid were calculated using gp23 or gp18 as the internal control (930 copies of gp23 and 138 copies of gp18 per capsid) and S-trimer protein standard.

### Example 11

### SUMO-RBD-Spy Protein Expression, Denaturing, Refolding, and Purification

The E. coli expression, denaturing, refolding, and purification of SUMO-RBD-Spy (rRBD) were performed²⁰. Briefly, the BL21-CodonPlus (DE3)-RIPL cells containing PET28b-SUMO-RBD-Spy were induced with 0.5 mM isopropyl--D-1-thiogalactopyranoside (IPTG) for 3 h at 28° C. Cells were harvested and resuspended in buffer A (20 mM Tris-HCl, 500 mM NaCl, 5 mM imidazole, 5 mM β-mercaptoethanol, pH 7.9) containing protease inhibitor cocktail (Roche®, USA, Indianapolis, Ind.) and Benzonase. After the cells were lysed using a French press (Aminco®, Urbana, Ill.) and centrifuged, the pellet containing the inclusion body proteins was resuspended and washed with buffer B (buffer A+0.5% Triton X-100). Then, the inclusion bodies were solubilized in buffer C (Buffer A+8 M urea) by incubating/stirring at 4° C. overnight, followed by centrifugation and clarification. The supernatant containing denaturing protein was loaded on HisTrap column (AKTA®-prime; GE® Healthcare) followed by buffer C washing. The rRBD was refolded on HisTrap column using a linear gradient urea buffer D (20 mM Tris-HCl, 500 mM NaCl, 5 mM imidazole, 1 mM GSH, 0.1 mM GSSG, 20% glycerol, pH 7.9) between 8 and 0 M. Finally, the column was washed with buffer E (20 mM Tris-HCl, 500 mM NaCl, 100 mM imidazole, 20% glycerol, 5% glucose, pH 7.9), and the refolding rRBD was eluted with buffer F (20 mM Tris-HCl, 500 mM NaCl, 800 mM imidazole, 20% glycerol, 5% glucose, pH 7.9) and dialyzed to remove imidazole. The proteins were then quantified, aliquoted, and stored at −80° C. until use.

### Example 12

### Western Blot Analysis

After treatment with multiple freeze-thaw cycles and Benzonase, phage particles were boiled in SDS loading buffer for 10 min, separated by 4-20% SDS-PAGE, and then transferred to nitrocellulose membrane PVDF (Bio-Rad®). The PVDF was then blocked with 5% bovine serum albumin (BSA)-PBS (pH 7.4) buffer at RT for 1 hr with gentle shaking. Anti-NP or anti-RBD primary antibodies were added to the blots and incubated overnight at 4° C. in PBS-5% BSA, followed by five times rinsing in PBST buffer [1 xPBS (pH 7.4) and 0.05% Tween 20]. Goat-anti-mouse or goat-anti-rabbit HRP-conjugated antibody (Thermo Fisher®) was applied at a 1:5000 dilution in 5% BSA-PBST for 1 hour at RT with gentle shaking. After rinsing five times in PBST, signals were visualized with an enhanced chemiluminescence substrate (Bio-Rad®) using the Bio-Rad® Gel Doc XR+ System and Image Lab software according to the manufacturer's instructions (Bio-Rad®).

### Example 13

### Transmission Electron Microscopy

The T4-SocA, T4-SpyCatcher, and T4-S trimer phages were applied to the carbon grid for 5 min at RT. The phage-loaded grid was frozen in liquid nitrogen using Gatan CP3 cryo-plunger. The cryo-electron microscopy images were collected and reconstructed by Zhiqing Wang at Purdue University using a Titan Krios microscope equipped with a charge-coupled device camera.

### Example 14

### Cell Culture and Transfection

HEK293T cells were maintained in Dulbecco's modified Eagle's medium (DMEM; Gibco®) supplemented with 1% antibiotics (Thermo Fisher®), 1×HEPES (Thermo Fisher®), and 10% fetal bovine serum (Thermo Fisher®). Cells were passaged with 0.25% (w/v) trypsin/0.53 mM EDTA at a subcultivation ratio of 1:5 at 80 to 90% confluence. Cultures were incubated in a humidified atmosphere at 37° C. and 5% CO₂. The plasmid containing human ACE2 (Addgene® #1786) was transfected into HEK293T cells using Lipofectamine® 2000 Transfection Reagent (Thermo Fisher®) according to the manufacturer's instructions. Two days after ACE2 plasmid transfection, the cells were used for RBD or phage binding assay.

### Example 15

### Measurement of the Inhibition of RBD Binding to Cell Surface ACE2 by Mice Sera

Human ACE2 transfected HEK293T cells were washed with PBS twice and then fixed with 4% formaldehyde for 15 mins at RT. After rinsing twice in PBS for 5 mins each, cells were blocked in blocking buffer (5% BSA-PBS) for 1 hr at RT. Recombinant SARS-CoV-2 RBD protein (Sino Biological®) was added to the cells to a final concentration of 0.2 μg/ml in the presence or absence of the sera with a series of dilutions. The unbound RBD was removed by washing the cells five times in PBST (PBS+0.1% Tween 20) for 5 mins each. The 1/1000 diluted human anti-RBD monoclonal antibody (Thermo Fisher®) was added to cells and incubated in a humidified chamber for 1 h at RT or overnight at 4° C. After rinsing five times in PBST for 5 mins each, Alexa® 488- or Rhodamine-conjugated goat anti-human secondary antibody was added (1/500 dilution) (Thermo Fisher®), and incubated for 2-3 hrs at RT in the dark. The cells were then rinsed five times in PBST for 5 mins each and counter-stained on 1 μg/ml Hoechst 33342 (Thermo Fisher®) for 5 mins. The fluorescent signals were observed by fluorescence microscopy (Carl Zeiss®).

### Example 16

### Measurement of the T4-S Trimer-GFP Phages Binding to Cell Surface ACE2

Soc-GFP protein was produced as described previouslyl⁹. Briefly, the recombinant Soc-GFP proteins were purified according to the basic protocol described as follows. The BL21 (DE3) RIPL cells harboring the recombinant clones were induced with 1 mM IPTG for 2 h at 30° C. The cells were harvested by centrifugation (4,000×g for 15 min at 4° C.) and resuspended in 50 mL of HisTrap binding buffer (50 mM Tris-HCl, pH 8.0, 20 mM imidazole, 300 mM NaCl). The cells were lysed using French-press (Aminco®) and the soluble fraction containing the His-tagged fusion protein was isolated by centrifugation at 34,000×g for 20 min. The supernatant was loaded onto a HisTrap column (GE® Healthcare) and washed with 50 mM imidazole containing buffer, and the protein was eluted with 20-500 mM linear imidazole gradient. The peak fractions were concentrated and purified by size exclusion chromatography using Hi-Load 16/60 Superdex®-200 (prep-grade) gel filtration column (GE® Healthcare) in a buffer containing 20 mM Tris-HCl (pH 8.0) and 100 mM NaCl. The peak fractions were concentrated and stored at ˜80° C.

In vitro display of Soc-GFP on the T4- SpyCatcher or T4-S trimer phage was assessed by the co-sedimentation, similar to the procedures of S trimer display on T4 phage. The T4-SpyCatcher-GFP or T4-S trimer-GFP phages were resuspended in Opti-MEM medium (Thermo Fisher®) and then added to ACE2-transfected HEK293T cells. After 6 hrs incubation, the unbound phages were removed by rinsing three times in PBS for 5 mins each. The GFP signals were observed by fluorescence microscopy (Carl Zeiss®).

### Example 17

### Mouse Immunizations

We followed the recommendation of the National Institutes of Health about mouse study (the Guide for the Care and Use of Laboratory Animals). All mouse experiments were approved by the Institutional Animal Care and Use Committee of the Catholic University of America (Washington, DC) (Office of Laboratory Animal Welfare assurance number A4431-01) and the University of Texas Medical Branch (Galveston, Tex.) (Office of Laboratory Animal Welfare assurance number A3314-01). The SARS-CoV-2 virus challenge study was conducted in the animal biosafety level 3 (ABSL3) suite, and the principal investigators have registered with the CDC to work with the virus.

Six- to eight-week-old female BALB/c mice (The Jackson Laboratory (JAX®)) were randomly grouped (5 mice per group) and allowed to acclimate for 14 days. Three times intramuscular immunizations were administrated into their hind legs with phage vaccine candidates. A total of 6 ×10¹¹ phages was injected on days 0 (prime), 21 (boost 1), and 42 (boost 2). Negative control mice received the same volume of PBS buffer (Naive) or the same amount of T4 control phage. A group of mice immunized with S trimer (20 μg) adjuvanted with Aluminum Alhydrogel was included as the positive control. Blood was drawn from each animal on days 0 (prebleed), 14, 35, and 56, and the isolated sera were stored at −80° C.

### Example 18

### Rabbit Immunizations

All experiments were performed at Envigo®/Cocalico Biologicals® (Reamstown, PA) in accordance with institutional guidelines. Adult New Zealand White rabbits were immunized i.m. in the flank region with 3×10¹¹ PFU T4 phages/dose in 0.2 mL saline (n =4 for each group). Pre-immune test-bleeds were first obtained via venipuncture of the marginal vein of the ear on Day 1. Animals were immunized on Days 1, and 15 (Prime+One-boost regimen). Immune sera were obtained on Day 25.

### Example 19

### ELISA Determination of IgG and IgG Subtype Antibodies

ELISA plates (Evergreen Scientific®) were coated with 100 μl per well of 1 μg/ml of SARS-CoV-2 S-ecto protein (Sino Biological®), SARS-CoV-2 RBD-untagged protein (Sino Biological®), SARS- CoV-2 NP protein (Sino Biological®), or SARS-CoV-2 E protein (1-75 aa) (Thermo Fisher®) in coating buffer [0.05 M sodium carbonate-sodium bicarbonate (pH 9.6)]. After overnight incubation at 4° C., the plates were washed two times with PBS buffer and blocked for 2 hr at 37° C. with 200 pl per well PBS-5% BSA buffer. Serum samples were diluted with a 5-fold dilution series beginning with an initial 100-fold dilution in PBS-1% BSA. One hundred microliters diluted serum samples were added to each well, and the plates were incubated at 37° C. for 1 hour. After washing five times with PBST (PBS+0.05% Tween-20), the secondary antibody was added at 1:10,000 dilution in PBS-1% BSA buffer (100 μl per well) using either goat-anti-mouse IgG-HRP, goat-anti-mouse IgG1-HRP, goat-anti-mouse IgG2a-HRP (Thermo Fisher®), or goat-anti-rabbit IgG-HRP (Abcam®). After incubation for 1 hour at 37° C. and five times washes with PBS-T buffer, plates were developed using the TMB (3,3′,5,5′-tetramethylbenzidine) Microwell Peroxidase Substrate System (KPL®). After 5-10 min, the enzymatic reaction was stopped by adding TMB BlueSTOP (KPL®) solution. The absorbance was read within 30 min at 650 nm on a VersaMax spectrophotometer. The endpoint titer was defined as the highest reciprocal dilution of serum to give an absorbance more than 2-fold of the mean background of the assay.

### Example 20

### Measurement of the Binding of T4 Displayed RBD or S-Trimer to Human ACE2 Protein

An ELISA to analyze the binding of rRBD, S-ecto-6P-spytag trimer, T4 displayed RBD/S-trimer to human ACE2 protein was performed similarly to the above described. Briefly, 100 ng protein or 1×10¹⁰ phages were coated on plates overnight at 4° C. After blocking with PBS-5% BSA buffer, recombinant human ACE2-mouse Fc protein (Sino Biological®) with a series of dilution was added and incubated for 1 hr at 37° C. Plates were then incubated with the secondary goat-anti-mouse IgG-HRP antibody and developed with TMB substrate. Reactions were stopped and the absorbance was measured at 650 nm on a VersaMax spectrophotometer.

### Example 21

Challenge of the Mice with Adapted Live SARS-CoV-2 Virus

Immunized mice were challenged with the MA SARS-CoV-2 MA10 strain, a gift from R. Baric at the University of North Carolina, by the intranasal route as previously described. Briefly, mice were inoculated with 60 ul of SARS-CoV-2 MA10 at a dose of about 10⁵ median tissue culture infectious dose. The animals were weighed every day over the indicated period of time for monitoring the onset of morbidity (weight loss and other signs of illness) and mortality, as the end points for evaluating the vaccine efficacy.

### Example 22

### Neutralization Assay of Live SARS-CoV-2

Neutralizing antibody titers in mouse immune sera were quantified by Vero E6 cell-based microneutralization assay using SARS-CoV-2 US-WA-1/2020 strain as previously described⁵⁰. Briefly, serially 1:3 downward diluted mouse sera that were decomplemented at 56° C. for 60 min in a 60-ul volume were incubated for 1 hour at RT in duplicate wells of 96-well microtiter plates that contained 120 infectious SARS-CoV-2 viruses in 60 ul in each well. After incubation under BSL-3 conditions, 100 ul of the mixtures in individual wells was transferred to Vero E6 cell monolayer grown in 96-well microtiter plates containing 100 ul of MEM and 2% fetal bovine serum medium in each well and was cultured for 72 hours at 37° C. before assessing the presence or absence of cytopathic effect (CPE). Neutralizing antibody titers of the tested specimens were calculated as the reciprocal of the highest dilution of sera that completely inhibited virus-induced CPE in at least 50% of the wells and expressed as 50% neutralizing titer.

Neutralizing antibody titers in rabbit immune sera were quantified using an automated, liquid handler-assisted, high-throughput, microfocus neutralization/high-content imaging methods developed at ViroVax. Briefly, rabbit sera (paired preimmune and immune) were first decomplemented at 56° C. for 60 min and were then serially diluted in 384-well plates, in duplicate, using a BioTek Precision 2000 liquid handler, along with two reference sera. Twenty-microliter aliquots of SARS-CoV-2 US-WA-1/2020 were added to all test wells and positive control wells to yield a final MOI of 10 under BSL-3 conditions. Vero (American Type Culture Collection®, CCL-81) cells were maintained in a high-glucose DMEM supplemented with 10% fetal bovine serum (HyClone® Laboratories, South Logan, Utah) and 1% penicillin/streptomycin at 37° C. with 5% CO₂. After preincubating the plates for 1 hour, 20 ul of Vero cells (10⁶/ml), containing propidium iodihdde (PI) (50 μg/ml), was added to all wells using the liquid handler. Plates were then loaded in an IncuCyte® S3 high-content imaging system (Essen BioScience/Sartorius®, Ann Arbor, Mich). Longitudinal image acquisition and processing for virus-induced CPE and cell death (PI uptake) were performed every 6 hours, until cell death profiles had crested and stabilized (3.5 days). Neutralizing antibody titers which were expressed as median inhibitory concentration (IC50) or IC90, were obtained from four-parameter logistic curve fits of cell death profiles using OriginPro 9 (OriginLab® Corp., Northampton, Mass.).

### Example 23

### Statistics

All the data were presented as means ±SEM except indicated. Statistical analyses were performed by Graph Pad® Prism 9.0 software using one-way or two-way Analysis of variance (ANOVA) according to the data. Tukey's multiple comparisons post-test was used to compare individual groups. Significant differences between two groups were indicated by *P<0.05, **P<0.01, ***P<0.001, and ****P<0.0001. ns, no significance. P-values of <0.05 were considered significant.

It is intended that the invention not be limited to the particular embodiment disclosed herein contemplated for carrying out this invention, but that the invention will include all embodiments falling within the scope of the claims.

All documents, patents, journal articles and other materials cited in the present application are incorporated herein by reference.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

### REFERENCES

The following references are referred to above and are incorporated herein by reference:

1. Hsieh, C. L. et al. Structure-based design of prefusion-stabilized SARS-CoV-2 spikes. Science 369, 1501-1505 (2020).

2. Ni, L. et al. Detection of SARS-CoV-2-Specific Humoral and Cellular Immunity in COVID-19 Convalescent Individuals. Immunity 52, 971-977 e973 (2020).

3. Abremski, K. & Black, L. W. The function of bacteriophage T4 internal protein I in a restrictive strain of Escherichia coli. Virology 97, 439-447 (1979).

4. Mullaney, J.M. & Black, L. W. Capsid targeting sequence targets foreign proteins into bacteriophage T4 and permits proteolytic processing. Journal of Molecular Biology 261, 372-385 (1996).

5. Sarkar, M. & Saha, S. Structural insight into the role of novel SARS-CoV-2 E protein: A potential target for vaccine development and other therapeutic strategies. PloS one 15, e0237300 (2020).

6. Lan, J. et al. Structure of the SARS-CoV-2 spike receptor-binding domain bound to the ACE2 receptor. Nature 581, 215-220 (2020).

7. Keeble, A. H. et al. Approaching infinite affinity through engineering of peptide-protein interaction. Proceedings of the National Academy of Sciences of the United States of America (2019).

8. Piccoli, L. et al. Mapping Neutralizing and Immunodominant Sites on the SARS-CoV-2 Spike Receptor-Binding Domain by Structure-Guided High-Resolution Serology. Cell 183, 1024-1042 e1021 (2020).

9. Robbiani, D. F. et al. Convergent antibody responses to SARS-CoV-2 in convalescent individuals. Nature 584, 437-442 (2020).

10. Bolles, M. et al. A double-inactivated severe acute respiratory syndrome coronavirus vaccine provides incomplete protection in mice and induces increased eosinophilic proinflammatory pulmonary response upon challenge. Journal of Virology 85, 12201-12215 (2011).

11. Jordan, M. B., Mills, D. M., Kappler, J., Marrack, P. & Cambier, J. C. Promotion of B cell immune responses via an alum-induced myeloid cell population. Science 304, 1808-1810 (2004).

12. Leist, S. R. et al. A Mouse-Adapted SARS-CoV-2 Induces Acute Lung Injury and Mortality in Standard Laboratory Mice. Cell 183, 1070-1085 e1012 (2020).

13. Tao, P., Wu, X., Tang, W. C., Zhu, J. & Rao, V. Engineering of Bacteriophage T4 Genome Using CRISPR-Cas9. ACS Synthetic Biology 6, 1952-1961 (2017).

14. Liu, Y. et al. Covalent modifications of bacteriophage genome confer a degree of resistance to bacterial CRISPR systems. Journal of Virology (2020).

15. Tao, P., Zhu, J., Mahalingam, M., Batra, H. & Rao, V.B. Bacteriophage T4 nanoparticles for vaccine delivery against infectious diseases. Advanced Drug Delivery Reviews (2018).

16. Kanekiyo, M., Ellis, D. & King, N. P. New Vaccine Design and Delivery Technologies. The Journal of Infectious Diseases 219, S88-S96 (2019).

17. Walls, A. C. et al. Elicitation of Potent Neutralizing Antibody Responses by Designed Protein Nanoparticle Vaccines for SARS-CoV-2. Cell 183, 1367-1382 e1317 (2020).

18. Zhu, J. et al. A prokaryotic-eukaryotic hybrid viral vector for delivery of large cargos of genes and proteins into human cells. Science Advances 5, eaax0064 (2019).

19. Tao, P. et al. In vitro and in vivo delivery of genes and proteins using the bacteriophage T4 DNA packaging machine. Proceedings of the National Academy of Sciences of the United States of America 110, 5846-5851 (2013).

20. Zhao, J. C., Zhao, Z. D., Wang, W. & Gao, X. M. Prokaryotic expression, refolding, and purification of fragment 450-650 of the spike protein of SARS-coronavirus. Protein Expression and Purification 39, 169-174 (2005).

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, products specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

While the present disclosure has been disclosed with references to certain embodiments, numerous modifications, alterations, and changes to the described embodiments are possible without departing from the sphere and scope of the present disclosure, as defined in the appended claims. Accordingly, it is intended that the present disclosure is not limited to the described embodiments, but that it has the full scope defined by the language of the following claims, and equivalents thereof.

What is claimed is:

1. A universal vaccine design platform comprising:
at least one bacterial phage; and
at least one host cell comprising:
at least one CRISPR plasmid; and
at least one donor plasmid;
wherein the bacterial phage can infect the host cell,
wherein the CRISPR plasmid comprises a gene encoding at least one endonuclease that can be expressed within the host cell and create a cut in the genome of the bacterial phage,
wherein the donor plasmid comprises at least one DNA segment that can be inserted into the genome of the bacterial phage at the cut created by the endonuclease encoded in the CRISPR plasmid, and
wherein the genome of the bacterial phage comprising at least one inserted DNA segment from the donor plasmid can be packaged and released from the host cell.

2. The universal vaccine design platform of claim 1, wherein the bacterial phage is T4 bacterial phage.

3. The universal vaccine design platform of claim 1, wherein the host cell is E. coli.

4. The universal vaccine design platform of claim 1, wherein the endonuclease encoded in the CRISPR plasmid is at least one selected from the group consisting Cas9 and Cpf1.

5. The universal vaccine design platform of claim 1, wherein the CRISPR plasmid further comprises a spacer sequence, wherein the determines the location of the cut created by the endonuclease.

6. The universal vaccine design platform of claim 1, wherein the DNA segment in the donor plasmid encodes full-length or portion of at least one component of SARS-CoV-2, wherein the component of SARS-CoV-2 is immunogenic.

7. The universal vaccine design platform of claim 6, wherein the component of SARS-CoV-2 is at least one selected from the group consisting of spike trimer, ectodomain of the spike trimer, the receptor binding domain (RBD) of the spike trimer, encolop (E) protein and nuceocapsid protein (NP).

8. The universal vaccine design platform of claim 2, wherein the DNA segment is at least one SARS-CoV-2 gene fused with Hoc or Soc genes, wherein the SARS-CoV-2 gene encodes full-length or portion of at least one protein component of SARS-CoV-2, and wherein the SARS-CoV-2 gene fused with Hoc or Soc genes can be expressed as a fusion protein that can be displayed on the surface of T4 phages.

9. The universal vaccine design platform of claim 2, wherein the DNA segment is at least one SARS-CoV-2 gene fused with capsid targeting sequence (CTS) at the N-terminal of the SARS-CoV-2 gene, wherein the SARS-CoV-2 gene encodes full-length or portion of at least one protein component of SARS-CoV-2, and wherein the SARS-CoV-2 gene fused with CTS can be expressed and packaged inside the recombinant T4 phages.

10. A method of producing vaccine comprising:
introducing at least one CRISPR plasmid and at least one donor plasmid into at least one host cell;
infecting the host cell with at least one bacterial phage; and
purifying the recombinant bacterial phage released from the host cell,
wherein the CRISPR plasmid comprises a gene encoding at least one endonuclease that can be expressed within the host cell and create a cut in the genome of the bacterial phage, and
wherein the donor plasmid comprises at least one DNA segment that can be inserted into the genome of the bacterial phage at the cut created by the endonuclease encoded in the CRISPR plasmid.

11. The method of claim 10, wherein the bacterial phage is T4 bacterial phage.

12. The method of claim 10, wherein the host cell is E. coli.

13. The method of claim 10, wherein the endonuclease encoded in the CRISPR plasmid is at least one selected from the group consisting Cas9 and Cpf1.

14. The method of claim 10, wherein the CRISPR plasmid further comprises a spacer sequence, wherein the determines the location of the cut created by the endonuclease.

15. The method of claim 10, wherein the DNA segment in the donor plasmid encodes full-length or a portion of at least one component of SARS-CoV-2.

16. The method of claim 11, wherein the recombinant bacterial phage is a spycatcher phage, wherein gene encoding spycatcher is fused with Hoc or Soc genes, inserted into T4 phage genome, and expressed as a fusion protein of spycatcher and Hoc or Soc, wherein the fusion protein is displayed on the surface of recombinant bacterial phage.

17. The method of claim 16, further comprising displaying at least one protein fused with spytag on the surface of the spycatcher phage through the binding between spytag and spycatcher.

18. A vaccine comprising at least one recombinant bacterial phage produced using the method of claim 10.

19. A vaccine comprising at least one recombinant bacterial phage produced using the universal vaccine design platform of claim 1.

20. A vaccine comprising at least one recombinant bacterial phage, wherein the recombinant bacterial phage comprises at least one modification selected from the group consisting of:
at least one gene encoding a component of SARS-CoV-2 inserted in the genome of T4 phage,
at least one component of SARS-CoV-2 displayed on the surface of T4 phage, and
at least one component of SARS-CoV-2 packaged in T4 phage but not inserted in the genome of T4 phage,
wherein the component of SARS-CoV-2 is immunogenic.

21. The vaccine of claim 20, wherein the vaccine is adjuvant free.

22. The vaccine of claim 20, wherein the component of SARS-CoV-2 is at least one selected from the group consisting of spike trimer, ectodomain of the spike trimer, the receptor binding domain (RBD) of the spike trimer, encolop (E) protein and nuceocapsid protein (NP).

23. The vaccine of claim 20, wherein the component of SARS-CoV-2 is displayed on the surface of T4 phage through at least one connecting mechanism selected from the group consisting of Soc, Hoc, and spytag-spycatcher crossbridge.

24. A universal vaccine design platform comprising:
at least one T4 bacterial phage; and
at least one E. coli host cell comprising:
at least one altered plasmid; and
at least one donor plasmid;
wherein the T4 bacterial phage can infect the E. coli host cell,
wherein the altered plasmid comprises a gene encoding at least one endonuclease that can be expressed within the E. coli host cell and create a cut in the genome of the T4 bacterial phage,
wherein the donor plasmid comprises at least one DNA segment that can be inserted into the genome of the T4 bacterial phage at the cut created by the endonuclease encoded in the altered plasmid, and
wherein the genome of the T4 bacterial phage comprising at least one inserted DNA segment from the donor plasmid can be packaged and released from the E. coli host cell.
